(19) United States
(12) Patent Application Publication
(10) Pub. No.: US 2012/0107892 A1
(43) Pub. Date: May 3, 2012

(54) PRODUCTION OF ETHANOL FROM LIGNOCELLULOSIC BIOMASS

(76) Inventors: Frank Agbogbo, Lebanon, NH (US); Jessica Johnson, South Amana, IA (US)

(21) Appl. No.: 13/139,824

(22) PCT Filed: Dec. 18, 2009
(86) PCT No.: PCT/US09/68741

§ 371 Date: Jan. 4, 2012

Related U.S. Application Data
(62) Division of application No. 61/139,714, filed on Dec. 22, 2008.

(51) Int. Cl. C12P 7/14

(52) U.S. Cl. 435/162

(57) ABSTRACT

Described herein are methods for converting lignocellulosic biomass to ethanol, comprising the step of contacting the lignocellulosic biomass with a mixture for a period of time at an initial temperature and an initial pH, wherein the mixture comprises a first microorganism and a second microorganism, thereby producing an amount of ethanol. The first microorganism or the second microorganism may be a thermophilic or mesophilic microorganism. The first microorganism may be a native cellulolytic microorganism or a native xylanolytic microorganism; and the second microorganism may be a genetically engineered xylanolytic microorganism or a genetically engineered cellulolytic microorganism. The microorganisms may be Clostridium thermocellum or Thermoanaerobacterium saccharolyticum, or any number of a wide variety of others.

### RELATED APPLICATIONS

This application claims the benefit of priority to U.S. Provisional Patent Application Ser. No. 61/139,714, filed Dec. 22, 2008; the contents of which are hereby incorporated by reference.

### BACKGROUND OF THE INVENTION

Energy conversion, utilization and access underlie many of the great challenges of our time, including those associated with sustainability, environmental quality, security, and poverty. New applications of emerging technologies are required to respond to these challenges. Biotechnology, one of the most powerful of the emerging technologies, can give rise to important new energy conversion processes.

Plant biomass and derivatives thereof are a resource for the biological conversion of energy to forms useful to humanity. Among forms of plant biomass, lignocellulosic biomass (“biomass”) is particularly well-suited for energy applications because of its large-scale availability, low cost, and environmentally benign production. In particular, many energy production and utilization cycles based on cellulosic biomass have near-zero greenhouse gas emissions on a life-cycle basis. The primary obstacle impeding the more widespread production of energy from biomass feedstocks is the general absence of low-cost technology for overcoming the recalcitrance of these materials to conversion into useful fuels. Lignocellulosic biomass contains carbohydrate fractions (e.g., cellulose and hemicellulose) that can be converted into ethanol. In order to convert these fractions to ethanol, the cellulose and hemicellulose must initially be converted or hydrolyzed into monosaccharides; this hydrolysis has historically proven to be problematic.

Biologically mediated processes are promising for energy conversion, in particular for the conversion of lignocellulosic biomass into fuels. Biomass processing schemes involving enzymatic or microbial hydrolysis commonly involve four biologically mediated transformations: (1) the production of saccharolytic enzymes (cellulases and hemicellulases); (2) the hydrolysis of carbohydrate components present in pretreated biomass to sugars; (3) the fermentation of hexose sugars (e.g., glucose, mannose, and galactose); and (4) the fermentation of pentose sugars (e.g., xylose and arabinose). These four transformations may occur in a single step in a process configuration called consolidated bioprocessing (CBP), which is distinguished from other less highly integrated configurations in that CBP does not involve a dedicated process step for cellulase and/or hemicellulase production.

CBP offers the potential for lower cost and higher efficiency than processes requiring dedicated cellulase production. The benefits result in part from avoided capital costs for substrate and other raw materials, and utilities associated with cellulase production. In addition, several factors support the realization of higher rates of hydrolysis, and hence reduced reactor volume and capital investment using CBP, including enzyme-microbe synergy and the use of thermophilic organisms and/or complexed cellulase systems. Moreover, cellulose-adherent cellulolytic microorganisms are likely to compete successfully for products of cellulose hydrolysis with non-adhered microbes, e.g., contaminants, which could increase the stability of industrial processes based on microbial cellulose utilization. Progress in developing CBP-enabling microorganisms is being made through two strategies: engineering naturally occurring cellulolytic and xylanolytic microorganisms to improve product-related properties, such as yield and titer; and engineering non-cellulolytic organisms that exhibit high product yields and titers to express a heterologous cellulase and hemicellulase system enabling cellulose and hemicellulose utilization.

Many bacteria have the ability to ferment simple hexose sugars into a mixture of acidic and pH-neutral products via the process of glycolysis. The glycolytic pathway is ubiquitous and comprises a series of enzymatic steps whereby a six carbon glucose molecule is broken down, via multiple intermediates, into two molecules of the three-carbon compound pyruvate. From this point, however, the pyruvate can be metabolized via several different pathways, only one of which produces ethanol. The majority of facultative anaerobic bacteria do not produce high yields of ethanol from pyruvate under either aerobic or anaerobic conditions. Most facultative anaerobes metabolize pyruvate aerobically via pyruvate dehydrogenase (PDH) and the tricarboxylic acid cycle (TCA), ultimately producing CO₂, water, and ATP. Under anaerobic conditions, the main energy pathway for the metabolism of pyruvate is via the pyruvate-formate-lyase (PFL) pathway to give formate and acetyl-CoA. Acetyl-CoA is then converted to acetate, via phosphotransacetylase (PTA) and acetate kinase (ACK) with the co-production of ATP, or reduced to ethanol via acetalaldehyde dehydrogenase (AcDH) and alcohol dehydrogenase (ADH). In order to maintain a balance of reducing equivalents, excess NADH produced from glycolysis is re-oxidized to NAD⁺ by lactate dehydrogenase (LDH) during the reduction of pyruvate to lactate. NADH can also be re-oxidized by AcDH and ADH during the reduction of acetyl-CoA to ethanol, but this pathway typically plays a minor role in cells with a functional LDH. Theoretical yields of ethanol, therefore, are not achieved because most acetyl CoA is converted to acetate to regenerate ATP, and excess NADH produced during glycolysis is oxidized by LDH.

Metabolic engineering of microorganisms could result in the creation of a targeted knockout of the genes encoding for the production of enzymes, such as lactate dehydrogenase. In this case, “knock out” of the genes means partial, substantial, or complete deletion, silencing, inactivation, or down-regulation. If the conversion of pyruvate to lactate (the salt form of lactic acid) by the action of LDH were not available in the early stages of the glycolytic pathway, then the pyruvate could be more efficiently converted to acetyl CoA by the action of pyruvate dehydrogenase or pyruvate-ferredoxin oxidoreductase. If the further conversion of acetyl CoA to acetate (the salt form of acetic acid) by phosphotransacetylase and acetate kinase were also unavailable, e.g., if the genes encoding for the production of PTA and ACK were knocked out, then the acetyl CoA could be more efficiently converted to ethanol by AcDH and ADH. Accordingly, a genetically-modified strain of microorganism with such targeted gene knockouts, which would decrease or eliminate the production of organic acids, should have an increased ability to produce ethanol as a fermentation product.

Ideally, desirable characteristics of different microorganisms could be utilized simultaneously by fermenting lignocellulosic biomass with co-cultures of the microorganisms. However, the optimal conditions for fermentation of lignocellulosic biomass vary greatly from species to species. Under the most favorable conditions, monocultures of bacteria can replicate very quickly and efficiently produce the desired fermentation product. However, due to evolutionary pressure, when a co-culture of microorganisms is present, the species that can grow the fastest often dominates. Many variables influence the success of bacterial fermentation of lignocellulosic biomass, including but not limited to: temperature, pH, growth medium, and pre-treatment protocol. Identifying the small window of conditions suitable for co-culturing at least two microorganisms, while the organisms simultaneously ferment lignocellulosic biomass, presents a significant challenge.

### SUMMARY OF THE INVENTION

In one embodiment, the invention relates to a method for converting lignocellulosic biomass to ethanol, comprising the step of contacting the lignocellulosic biomass with a mixture for a period of time at an initial temperature and an initial pH, wherein the mixture comprises a first microorganism and a second microorganism, thereby producing an amount of ethanol. In certain embodiments, the first microorganism is a thermophilic or mesophilic microorganism. In certain embodiments, the second microorganism is a thermophilic or mesophilic microorganism.

In certain embodiments, the first microorganism is a cellulolytic microorganism. In certain embodiments, the second microorganism is a xylanolytic microorganism. In certain embodiments, the first microorganism is a cellulolytic microorganism; and the second microorganism is a xylanolytic microorganism. In certain embodiments, the first microorganism is a native cellulolytic microorganism. In certain embodiments, the second microorganism is a genetically engineered xylanolytic microorganism. In certain embodiments, the first microorganism is a native cellulolytic microorganism; and the second microorganism is a genetically engineered xylanolytic microorganism. In certain embodiments, the first microorganism is native Clostridium thermocellum. In certain embodiments, the second microorganism is a genetically engineered Thermoanaerobacterium saccharolyticum. In certain embodiments, the first microorganism is native Clostridium thermocellum; and the second microorganism is a genetically engineered Thermoanaerobacterium saccharolyticum.

In certain embodiments, the first microorganism is a xylanolytic microorganism. In certain embodiments, the second microorganism is a cellulolytic microorganism. In certain embodiments, the first microorganism is a xylanolytic microorganism; and the second microorganism is a cellulolytic microorganism. In certain embodiments, the first microorganism is a native xylanolytic microorganism. In certain embodiments, the second microorganism is a genetically engineered cellulolytic microorganism. In certain embodiments, the first microorganism is a native xylanolytic microorganism; and the second microorganism is a genetically engineered cellulolytic microorganism. In certain embodiments, the first microorganism is native Thermoanaerobacterium saccharolyticum. In certain embodiments, the second microorganism is a genetically engineered Clostridium thermocellum. In certain embodiments, the first microorganism is native Thermoanaerobacterium saccharolyticum; and the second microorganism is a genetically engineered Clostridium thermocellum.

In one embodiment, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is native Clostridium thermocellum; and the second microorganism is Thermoanaerobacterium saccharolyticum. In certain embodiments, the second microorganism is a genetically-modified Thermoanaerobacterium saccharolyticum.

In one embodiment, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is native Thermoanaerobacterium saccharolyticum; and the second microorganism is Clostridium thermocellum. In certain embodiments, the second microorganism is a genetically-modified Clostridium thermocellum.

### BRIEF DESCRIPTION OF THE DRAWINGS

FIG. 1 depicts the fermentation product profiles of: (a) a co-culture of C. thermocellum with engineered T. saccharolyticum adapted to pH 7 and 60° C. (represented by squares) on 5% mixed hardwoods in serum bottles; and (b) C. thermocellum (represented by diamonds) monoculture on 5% mixed hardwoods in serum bottles.

FIG. 2 depicts a total product profile comparison of: (a) a co-culture of C. thermocellum with engineered T. saccharolyticum adapted to pH 7 and 60° C. on 2% unwashed mixed hardwoods (represented by X); (b) a co-culture of C. thermocellum with engineered T. saccharolyticum adapted to pH 7 and 60° C. on 2% washed mixed hardwoods (represented by squares); (c) C. thermocellum monoculture on 2% unwashed mixed hardwoods (represented by triangles); and (d) C. thermocellum monoculture on 2% washed mixed hardwoods (represented by diamonds).

FIG. 3 depicts an ethanol yield comparison of: (a) a co-culture of C. thermocellum with engineered T. saccharolyticum adapted to pH 7 and 60° C. on 2% unwashed mixed hardwoods (represented by X); (b) a co-culture of C. thermocellum with engineered T. saccharolyticum adapted to pH 7 and 60° C. on 2% washed mixed hardwoods (represented by squares); (c) C. thermocellum monoculture on 2% unwashed mixed hardwoods (represented by triangles); and (d) C. thermocellum monoculture on 2% washed mixed hardwoods (represented by diamonds).

FIG. 4 depicts growth curves as a function of time for: (a) unadapted T. saccharolyticum (MO 355) at 60° C. and pH 7 (represented by diamonds); (b) pH-adapted T. saccharolyticum (MO 521) at 60° C. and pH 7 (represented by squares); (c) pH-adapted T. saccharolyticum (MO 699) at 60° C. and pH 7 (represented by triangles); (d) pH-adapted T. saccharolyticum (MO 694) at 60° C. and pH 7 (represented by X); and (e) pH-adapted T. saccharolyticum (MO 728) at 60° C. and pH 7 (represented by asterisks).

FIG. 5 depicts ethanol yields obtained with T. saccharolyticum alone, C. thermocellum LDH KO 1313 alone, and C. thermocellum strains 27405 and LDH KO 1313, respectively, co-cultured with T. saccharolyticum.

FIG. 6 depicts total product yields obtained with T. saccharolyticum alone, C. thermocellum LDH KO 1313 alone, and C. thermocellum strains 27405 and LDH KO 1313, respectively, co-cultured with T. saccharolyticum.

FIG. 7 depicts variation of ethanol, acetate and total yield on 20 g/L Avicel of the residue that remains after yeast fermentation.

FIG. 8 depicts the fermentation profile of 80 g/L Avicel in a co-culture at 55° C. and pH 6.

FIG. 9 depicts the fermentation profile for a co-culture fermentation on 160 g/L Avicel.

FIG. 10 depicts ethanol concentration and yields at various Avicel concentrations.

FIG. 11 depicts the total product yield from unwashed MS 149 (milled and unmilled) at 20 g/L solids.

FIG. 12 depicts the theoretical ethanol yield from unwashed MS 149 (milled and unmilled) at 20 g/L solids.

FIG. 13 depicts the product distribution from unwashed MS 149 (milled and unmilled) at 20 g/L solids.

FIG. 14 depicts ethanol concentrations from 2-7.5% unwashed solids at times from 236 h to 400 h.

FIG. 15 depicts ethanol concentrations from 2-7.5% washed solids at times from 236 h to 400 h.

FIG. 16 depicts ethanol yields from 2-7.5% washed solids at times from 236 h to 400 h.

FIG. 17 depicts the final product concentrations from 5% MS 419 using a monoculture and co-culture.

FIG. 18 depicts product distribution from paper sludge at 100 g/L solids.

FIG. 19 depicts the product concentrations from 14.9% unwashed mixed hardwoods and 3% paper sludge in a co-culture.

FIG. 20 depicts results from a stable, mutualistic consortium co-culture intentionally contaminated with Geobacillus thermoglucosidiasus.

FIG. 21 depicts the product concentrations from mono- and co-cultures of C. thermocellum and T. thermosaccharolyticum.

FIG. 22 depicts product concentrations and yields on the transfer of co-cultures on Avicel, xylan, and xylose.

FIG. 23 depicts a comparison of the production of ethanol and exopolysaccharides (EPS) from a monoculture and co-culture.

### DETAILED DESCRIPTION OF THE INVENTION

### Overview

Aspects of the present invention relate to a process by which the efficiency and cost of ethanol production from cellulosic biomass-containing materials can be reduced by using a novel consolidated bioprocessing (CBP) methodology. In particular, the present invention provides numerous methods for increasing the efficiency of ethanol production from biomass by microorganisms.

One aspect of the invention relates to a method for the conversion of lignocellulosic biomass into ethanol utilizing co-cultures of at least two microorganisms. By exploiting certain desirable characteristics from each organism in the co-culture, unexpectedly high levels of ethanol are produced in comparison to the levels of ethanol produced in monocultures of the individual microorganisms. For example, because substrates often contain cellulose and xylan (hemi-cellulose) components, a microorganism capable of utilizing cellulose is combined with a microorganism capable of utilizing xylan in certain embodiments of the invention. In this respect, the efforts of the microorganisms are orthogonal, but complementary. Processes utilizing co-cultures, therefore, offer significant benefits over standard monoculture-based processes.

By virtue of a novel integration of processing steps, commonly known as consolidated bioprocessing, aspects of the present invention provide for more efficient production of ethanol from cellulosic-biomass-containing raw materials. One of the leading economic challenges in converting biomass to ethanol is the cost of additional enzymes that are typically added to the broth. One aspect of the present inventions provides for a process in which no external enzymes are added, thus making the process extremely cost-effective. Additionally, the incorporation of genetically-modified thermophilic or mesophilic microorganisms in the processing of said materials allows for fermentation steps to be conducted at higher temperatures, thereby improving process economics. For example, reaction kinetics are typically a function of temperature, so higher temperatures are generally associated with increases in the overall rate of production. Additionally, higher temperatures facilitate the removal of volatile products from the broth, and reduces the need for cooling of the substrate after pretreatment (a preceding step that is typically conducted at an elevated temperature).

Operating CBP processes at thermophilic temperatures offers several important benefits over conventional mesophilic fermentation temperatures of 30-37° C. In particular, costs associated with having a process step dedicated to cellulase production are eliminated for CBP. Costs associated with fermentor cooling and heat-exchange before and after fermentation are also expected to be reduced for CBP. Moreover, processes featuring thermophilic biocatalysts may be less susceptible to microbial contamination as compared to processes featuring conventional mesophilic biocatalysts. Additionally, combining strains of complementary microorganisms that are good at utilizing cellulose and xylan, respectively, and producing ethanol as the major product renders the processes more economical than current industrial processes because, for example, a greater proportion of the biomass is converted to ethanol, and added enzymes are not needed.

In one embodiment, the present invention provides for a method of converting to ethanol hardwoods pretreated by autohydrolysis via fermentation with a co-culture of a cellulolytic and xylanolytic microorganisms, without the use of exogenous enzymes.

### DEFINITIONS

The term “expression” is intended to include the expression of a gene at least at the level of mRNA production.

The term “expression product” is intended to include the resultant product, e.g., a polypeptide, of an expressed gene.

The term “increased expression” is intended to include an alteration in gene expression at least at the level of increased mRNA production and, preferably, at the level of polypeptide expression. The term “increased production” is intended to include an increase in the amount of a polypeptide expressed, in the level of the enzymatic activity of the polypeptide, or a combination thereof.

The terms “activity,” “activities,” “enzymatic activity,” and “enzymatic activities” are used interchangeably and are intended to include any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof. Techniques for determining total activity as compared to secreted activity are described herein and are known in the art.

The term “xylanolytic activity” is intended to include the ability to hydrolyze glycosidic linkages in oligopentoses and polypentoses.

The term “cellulolytic activity” is intended to include the ability to hydrolyze partially, substantially or completely cellulose or any of its constituents. Cellulolytic activity may also include the ability to depolymerize or debranch cellulose and hemicellulose.

The term “xylanolytic activity” is intended to include the ability to hydrolyze glycosidic linkages in oligopentoses and polypentoses.

As used herein, the term “lactate dehydrogenase” or “LDH” is intended to include the enzyme capable of converting pyruvate into lactate. It is understood that LDH can also catalyze the oxidation of hydroxybutyrate.

As used herein the term “alcohol dehydrogenase” or “ADH” is intended to include the enzyme capable of converting acetaldehyde into an alcohol, advantageously, ethanol.

The term “pyruvate decarboxylase activity” is intended to include the ability of a polypeptide to enzymatically convert pyruvate into acetaldehyde (e.g., “pyruvate decarboxylase” or “PDC”). Typically, the activity of a selected polypeptide encompasses the total enzymatic activity associated with the produced polypeptide, comprising, e.g., the superior substrate affinity of the enzyme, thermostability, stability at different pHs, or a combination of these attributes.

The term “ethanologenic” is intended to include the ability of a microorganism to produce ethanol from a carbohydrate as a fermentation product. The term is intended to include, but is not limited to, naturally occurring ethanologenic organisms, ethanologenic organisms with naturally occurring or induced mutations, and ethanologenic organisms which have been genetically modified.

The terms “fermenting” and “fermentation” are intended to include the enzymatic process (e.g., cellular or acellular, e.g., a lysate or purified polypeptide mixture) by which ethanol is produced from a carbohydrate, in particular, as a product of fermentation.

By “thermophilic” is meant an organism that thrives at a temperature of about 45° C. or higher.

By “mesophilic” is meant an organism that thrives at a temperature of about 20° C.-45° C.

The term “organic acid” is art-recognized. The term “lactic acid” refers to the organic acid 2-hydroxypropionic acid in either the free acid or salt form. The salt form of lactic acid is referred to as “lactate” regardless of the neutralizing agent, i.e., calcium carbonate or ammonium hydroxide. The term “acetic acid” refers to the organic acid methanecarboxylic acid, also known as ethanoic acid, in either free acid or salt form. The salt form of acetic acid is referred to as “acetate.”

The terms “lignocellulosic material,” “lignocellulosic substrate,” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants, and sugar-processing residues.

The term “co-culture” means a mixture of at least two microorganisms that have been reproduced in predetermined culture media under controlled laboratory conditions, either together or separately.

### Exemplary Methods

Aspects of the present invention relate to methods useful in the production of ethanol from lignocellulosic biomass substrates.

In one embodiment, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism. In certain embodiments, the first microorganism is a thermophilic or mesophilic microorganism. In certain embodiments, the second microorganism is a thermophilic or mesophilic microorganism.

In certain embodiments, the first microorganism is a cellulolytic microorganism. In certain embodiments, the second microorganism is a xylanolytic microorganism. In certain embodiments, the first microorganism is a cellulolytic microorganism; and the second microorganism is a xylanolytic microorganism. In certain embodiments, the first microorganism is a native cellulolytic microorganism. In certain embodiments, the second microorganism is a genetically engineered xylanolytic microorganism. In certain embodiments, the first microorganism is a native cellulolytic microorganism; and the second microorganism is a genetically engineered xylanolytic microorganism. In certain embodiments, the first microorganism is native Clostridium thermocellum. In certain embodiments, the second microorganism is a genetically engineered Thermoanaerobacterium saccharolyticum. In certain embodiments, the first microorganism is native Clostridium thermocellum; and the second microorganism is a genetically engineered Thermoanaerobacterium saccharolyticum.

In certain embodiments, the invention relates to any one of the above-mentioned methods, wherein the first microorganism is a xylanolytic microorganism. In certain embodiments, the second microorganism is a cellulolytic microorganism. In certain embodiments, the first microorganism is a xylanolytic microorganism; and the second microorganism is a cellulolytic microorganism. In certain embodiments, the first microorganism is a native xylanolytic microorganism. In certain embodiments, the second microorganism is a genetically engineered cellulolytic microorganism. In certain embodiments, the first microorganism is a native xylanolytic microorganism; and the second microorganism is a genetically engineered cellulolytic microorganism. In certain embodiments, wherein the first microorganism is native Thermoanaerobacterium saccharolyticum. In certain embodiments, the second microorganism is a genetically engineered Clostridium thermocellum. In certain embodiments, wherein the first microorganism is native Thermoanaerobacterium saccharolyticum; and the second microorganism is a genetically engineered Clostridium thermocellum.

In certain embodiments, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises at least two microorganisms; and at least one of the microorganisms comprises at least one genetic modification. In certain embodiments, the invention relates to a method utilizing one or more genetically-modified thermophilic or mesophilic microorganisms comprising a gene or a particular polynucleotide sequence that has been partially, substantially, or completely deleted, silenced, inactivated, or down-regulated, which gene or polynucleotide sequence encodes for an enzyme that confers upon the microorganism the ability to produce organic acids as fermentation products; thereby increasing the ability of the microorganism to produce ethanol as a fermentation product.

In certain embodiments, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises at least two microorganisms; and at least one of the microorganisms comprises at least one genetic modification. In certain embodiments, the invention relates to a method utilizing one or more genetically-modified thermophilic or mesophilic microorganisms, wherein (a) a first native gene has been partially, substantially, or completely deleted, silenced, inactivated, or down-regulated, which first native gene encodes a first native enzyme involved in the metabolic production of an organic acid or a salt thereof, and (b) a first non-native gene has been inserted, which first non-native gene encodes a first non-native enzyme involved in the metabolic production of ethanol; thereby increasing the ability of said thermophilic or mesophilic microorganism to produce ethanol as a fermentation product.

In certain embodiments, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises at least two microorganisms; and at least one of the microorganisms comprises at least one genetic modification. In certain embodiments, the invention relates to a method utilizing one or more genetically-modified thermophilic or mesophilic microorganisms, wherein (a) a first native gene has been partially, substantially, or completely deleted, silenced, inactivated, or down-regulated, which first native gene encodes a first native enzyme involved in the metabolic production of an organic acid or a salt thereof, and (b) a first non-native gene has been inserted, which first non-native gene encodes a first non-native enzyme involved in the hydrolysis of a polysaccharide; thereby increasing the ability of said thermophilic or mesophilic microorganism to produce ethanol as a fermentation product.

In certain embodiments, the invention relates to any one of the above-mentioned methods, wherein the amount of ethanol produced is at least about 60% of the theoretical yield based on the amount of lignocellulosic biomass metabolized.

In certain embodiments, the invention relates to any one of the above-mentioned methods, wherein the amount of ethanol produced is at least about 70% of the theoretical yield based on the amount of lignocellulosic biomass metabolized.

In certain embodiments, the invention relates to any one of the above-mentioned methods, wherein the amount of ethanol produced is at least about 80% of the theoretical yield based on the amount of lignocellulosic biomass metabolized.

In certain embodiments, the invention relates to any one of the above-mentioned methods, wherein the amount of ethanol produced is at least about 90% of the theoretical yield based on the amount of lignocellulosic biomass metabolized.

In certain embodiments, the invention relates to any one of the above-mentioned methods, wherein the period of time is about 10 hours to about 300 hours.

In certain embodiments, the invention relates to any one of the above-mentioned methods, wherein the period of time is about 50 hours to about 200 hours.

In certain embodiments, the invention relates to any one of the above-mentioned methods, wherein the period of time is about 80 hours to about 160 hours.

In certain embodiments, the invention relates to any one of the above-mentioned methods, wherein the period of time is about 80 hours (h), about 85 h, about 90 h, about 95 h, about 100 h, about 105 h, about 110 h, about 115 h, about 120 h, about 125 h, about 130 h, about 135 h, about 140 h, about 145 h, about 150 h, about 155 h, or about 160 h.

In certain embodiments, the invention relates to any one of the above-mentioned methods, wherein the period of time is about 120 hours.

In certain embodiments, the invention relates to any one of the above-mentioned methods, wherein the initial temperature is about 30° C. to about 75° C.

In certain embodiments, the invention relates to any one of the above-mentioned methods, wherein the initial temperature is about 45° C. to about 75° C.

In certain embodiments, the invention relates to any one of the above-mentioned methods, wherein the initial temperature is about 55° C. to about 65° C.

In certain embodiments, the invention relates to any one of the above-mentioned methods, wherein the initial temperature is about 60° C.

In certain embodiments, the invention relates to any one of the above-mentioned methods, wherein the initial pH is between about 5 and about 9.

In certain embodiments, the invention relates to any one of the above-mentioned methods, wherein the initial pH is between about 6 and about 8.

In certain embodiments, the invention relates to any one of the above-mentioned methods, wherein the initial pH is about 5, about 5.5, about 6, about 6.5, about 7, about 7.5, about 8, about 8.5, or about 9.

In certain embodiments, the invention relates to any one of the above-mentioned methods, wherein the initial pH is about 6, about 6.5, about 7, about 7.5, or about 8.

In certain embodiments, the invention relates to any one of the above-mentioned methods, wherein the initial pH is about 7 or about 7.5.

In one embodiment, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is selected from the group consisting of Clostridium thermocellum, Clostridium cellulolyticum, Thermoanaerobacterium saccharolyticum, Clostridium stercorarium, Clostridium stercorarium II, Caldiscellulosiruptor kristjanssonii, and Clostridium phytofermentans; and the second microorganism is selected from the group consisting of Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellum thermophilum.

In certain embodiments, the invention relates to any one of the above-mentioned methods, wherein the first microorganism is Clostridium thermocellum; and the second microorganism is Thermoanaerobacterium saccharolyticum.

In certain embodiments, the invention relates to any one of the above-mentioned methods, wherein the first microorganism or the second microorganism comprises at least one genetic modification.

In certain embodiments, the invention relates to any one of the above-mentioned methods, wherein the second microorganism comprises at least one genetic modification.

In certain embodiments, the invention relates to any one of the above-mentioned methods, wherein the first microorganism comprises at least one genetic modification.

In certain embodiments, the invention relates to any one of the above-mentioned methods, wherein at least one of the microorganisms is a genetically-modified thermophilic or mesophilic microorganism comprising a gene or a particular polynucleotide sequence that has been partially, substantially, or completely deleted, silenced, inactivated, or down-regulated, which gene or polynucleotide sequence encodes for an enzyme that confers upon the microorganism the ability to produce organic acids as fermentation products; thereby increasing the ability of the microorganism to produce ethanol as a fermentation product.

In certain embodiments, the invention relates to any one of the above-mentioned methods, wherein at least one of the microorganisms is a genetically-modified thermophilic or mesophilic microorganism comprising (a) a first native gene that has been partially, substantially, or completely deleted, silenced, inactivated, or down-regulated, which first native gene encodes a first native enzyme involved in the metabolic production of an organic acid or a salt thereof, and (b) a first non-native gene that has been inserted, which first non-native gene encodes a first non-native enzyme involved in the metabolic production of ethanol; thereby increasing the ability of said thermophilic or mesophilic microorganism to produce ethanol as a fermentation product.

In certain embodiments, the invention relates to any one of the above-mentioned methods, wherein at least one of the microorganisms is a genetically-modified thermophilic or mesophilic microorganism comprising (a) a first native gene that has been partially, substantially, or completely deleted, silenced, inactivated, or down-regulated, which first native gene encodes a first native enzyme involved in the metabolic production of an organic acid or a salt thereof, and (b) a first non-native gene that has been inserted, which first non-native gene encodes a first non-native enzyme involved in the hydrolysis of a polysaccharide; thereby increasing the ability of said thermophilic or mesophilic microorganism to produce ethanol as a fermentation product.

In certain embodiments, the invention relates to any one of the above-mentioned methods, wherein the amount of ethanol produced is at least about 60% of the theoretical yield based on the amount of lignocellulosic biomass metabolized.

In certain embodiments, the invention relates to any one of the above-mentioned methods, wherein the amount of ethanol produced is at least about 70% of the theoretical yield based on the amount of lignocellulosic biomass metabolized.

In certain embodiments, the invention relates to any one of the above-mentioned methods, wherein the amount of ethanol produced is at least about 80% of the theoretical yield based on the amount of lignocellulosic biomass metabolized.

In certain embodiments, the invention relates to any one of the above-mentioned methods, wherein the amount of ethanol produced is at least about 90% of the theoretical yield based on the amount of lignocellulosic biomass metabolized.

In certain embodiments, the invention relates to any one of the above-mentioned methods, wherein the period of time is about 10 hours to about 300 hours.

In certain embodiments, the invention relates to any one of the above-mentioned methods, wherein the period of time is about 50 hours to about 200 hours.

In certain embodiments, the invention relates to any one of the above-mentioned methods, wherein the period of time is about 80 hours to about 160 hours.

In certain embodiments, the invention relates to any one of the above-mentioned methods, wherein the period of time is about 80 hours (h), about 85 h, about 90 h, about 95 h, about 100 h, about 105 h, about 110 h, about 115 h, about 120 h, about 125 h, about 130 h, about 135 h, about 140 h, about 145 h, about 150 h, about 155 h, or about 160 h.

In certain embodiments, the invention relates to any one of the above-mentioned methods, wherein the period of time is about 120 hours.

In certain embodiments, the invention relates to any one of the above-mentioned methods, wherein the initial temperature is about 30° C. to about 75° C.

In certain embodiments, the invention relates to any one of the above-mentioned methods, wherein the initial temperature is about 45° C. to about 75° C.

In certain embodiments, the invention relates to any one of the above-mentioned methods, wherein the initial temperature is about 55° C. to about 65° C.

In certain embodiments, the invention relates to any one of the above-mentioned methods, wherein the initial temperature is about 60° C.

In certain embodiments, the invention relates to any one of the above-mentioned methods, wherein the initial pH is between about 5 and about 9.

In certain embodiments, the invention relates to any one of the above-mentioned methods, wherein the initial pH is between about 6 and about 8.

In certain embodiments, the invention relates to any one of the above-mentioned methods, wherein the initial pH is about 5, about 5.5, about 6, about 6.5, about 7, about 7.5, about 8, about 8.5, or about 9.

In certain embodiments, the invention relates to any one of the above-mentioned methods, wherein the initial pH is about 6, about 6.5, about 7, about 7.5, or about 8.

In certain embodiments, the invention relates to any one of the above-mentioned methods, wherein the initial pH is about 7 or about 7.5.

In one embodiment, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is Clostridium thermocellum; and the second microorganism is Thermoanaerobacterium saccharolyticum.

In certain embodiments, the invention relates to the aforementioned method, wherein the second microorganism comprises at least one genetic modification.

In certain embodiments, the invention relates to the aforementioned method utilizing a genetically-modified second microorganism comprising a gene or a particular polynucleotide sequence that has been partially, substantially, or completely deleted, silenced, inactivated, or down-regulated, which gene or polynucleotide sequence encodes for an enzyme that confers upon the microorganism the ability to produce organic acids as fermentation products; thereby increasing the ability of the microorganism to produce ethanol as a fermentation product.

In certain embodiments, the invention relates to the aforementioned method utilizing a genetically-modified second microorganism comprising (a) a first native gene that has been partially, substantially, or completely deleted, silenced, inactivated, or down-regulated, which first native gene encodes a first native enzyme involved in the metabolic production of an organic acid or a salt thereof, and (b) a first non-native gene that has been inserted, which first non-native gene encodes a first non-native enzyme involved in the metabolic production of ethanol; thereby increasing the ability of said thermophilic or mesophilic microorganism to produce ethanol as a fermentation product.

In certain embodiments, the invention relates to the aforementioned method utilizing a genetically-modified second microorganism comprising (a) a first native gene that has been partially, substantially, or completely deleted, silenced, inactivated, or down-regulated, which first native gene encodes a first native enzyme involved in the metabolic production of an organic acid or a salt thereof, and (b) a first non-native gene that has been inserted, which first non-native gene encodes a first non-native enzyme involved in the hydrolysis of a polysaccharide; thereby increasing the ability of said thermophilic or mesophilic microorganism to produce ethanol as a fermentation product.

In certain embodiments, the invention relates to any one of the above-mentioned methods, wherein the genetically-modified first microorganism comprises a gene or a particular polynucleotide sequence that has been partially, substantially, or completely deleted, silenced, inactivated, or down-regulated, which gene or polynucleotide sequence encodes for an enzyme that confers upon the microorganism the ability to produce organic acids as fermentation products; thereby increasing the ability of the microorganism to produce ethanol as a fermentation product.

In certain embodiments, the invention relates to any one of the above-mentioned methods, wherein the genetically-modified first microorganism comprises (a) a first native gene that has been partially, substantially, or completely deleted, silenced, inactivated, or down-regulated, which first native gene encodes a first native enzyme involved in the metabolic production of an organic acid or a salt thereof; and (b) a first non-native gene that has been inserted, which first non-native gene encodes a first non-native enzyme involved in the metabolic production of ethanol; thereby increasing the ability of the microorganism to produce ethanol as a fermentation product.

In certain embodiments, the invention relates to any one of the above-mentioned methods, wherein the genetically-modified first microorganism comprises (a) a first native gene that has been partially, substantially, or completely deleted, silenced, inactivated, or down-regulated, which first native gene encodes a first native enzyme involved in the metabolic production of an organic acid or a salt thereof, and (b) a first non-native gene that has been inserted, which first non-native gene encodes a first non-native enzyme involved in the hydrolysis of a polysaccharide; thereby increasing the ability of the microorganism to produce ethanol as a fermentation product.

In certain embodiments, the invention relates to any one of the above-mentioned methods, wherein the amount of ethanol produced is at least about 60% of the theoretical yield based on the amount of lignocellulosic biomass metabolized.

In certain embodiments, the invention relates to any one of the above-mentioned methods, wherein the amount of ethanol produced is at least about 70% of the theoretical yield based on the amount of lignocellulosic biomass metabolized.

In certain embodiments, the invention relates to any one of the above-mentioned methods, wherein the amount of ethanol produced is at least about 80% of the theoretical yield based on the amount of lignocellulosic biomass metabolized.

In certain embodiments, the invention relates to any one of the above-mentioned methods, wherein the amount of ethanol produced is at least about 90% of the theoretical yield based on the amount of lignocellulosic biomass metabolized.

In certain embodiments, the invention relates to any one of the above-mentioned methods, wherein the period of time is about 10 hours to about 300 hours.

In certain embodiments, the invention relates to any one of the above-mentioned methods, wherein the period of time is about 50 hours to about 200 hours.

In certain embodiments, the invention relates to any one of the above-mentioned methods, wherein the period of time is about 80 hours to about 160 hours.

In certain embodiments, the invention relates to any one of the above-mentioned methods, wherein the period of time is about 80 hours (h), about 85 h, about 90 h, about 95 h, about 100 h, about 105 h, about 110 h, about 115 h, about 120 h, about 125 h, about 130 h, about 135 h, about 140 h, about 145 h, about 150 h, about 155 h, or about 160 h.

In certain embodiments, the invention relates to any one of the above-mentioned methods, wherein the period of time is about 120 hours.

In certain embodiments, the invention relates to any one of the above-mentioned methods, wherein the initial temperature is about 30° C. to about 75° C.

In certain embodiments, the invention relates to any one of the above-mentioned methods, wherein the initial temperature is about 45° C. to about 75° C.

In certain embodiments, the invention relates to any one of the above-mentioned methods, wherein the initial temperature is about 55° C. to about 65° C.

In certain embodiments, the invention relates to any one of the above-mentioned methods, wherein the initial temperature is about 60° C.

In certain embodiments, the invention relates to any one of the above-mentioned methods, wherein the initial pH is between about 5 and about 9.

In certain embodiments, the invention relates to any one of the above-mentioned methods, wherein the initial pH is between about 6 and about 8.

In certain embodiments, the invention relates to any one of the above-mentioned methods, wherein the initial pH is about 5, about 5.5, about 6, about 6.5, about 7, about 7.5, about 8, about 8.5, or about 9.

In certain embodiments, the invention relates to any one of the above-mentioned methods, wherein the initial pH is about 6, about 6.5, about 7, about 7.5, or about 8.

In certain embodiments, the invention relates to any one of the above-mentioned methods, wherein the initial pH is about 7 or about 7.5.

In one embodiment, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is Clostridium thermocellum; the second microorganism is Thermoanaerobacterium saccharolyticum; the period of time is about 120 h, the initial temperature is about 60° C.; and the initial pH is about 7 or 7.5.

In certain embodiments, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is Clostridium thermocellum; the second microorganism is Thermoanaerobacterium saccharolyticum; the ack gene of the Thermoanaerobacterium saccharolyticum has been partially, substantially, or completely deleted, silenced, inactivated, or down-regulated, thereby producing a genetically-modified Thermoanaerobacterium saccharolyticum; the initial temperature is about 60° C.; and the initial pH is about 7.

In certain embodiments, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is Thermoanaerobacterium saccharolyticum; the second microorganism is Clostridium thermocellum; the ldh gene of the Clostridium thermocellum has been partially, substantially, or completely deleted, silenced, inactivated, or down-regulated, thereby producing a genetically-modified Clostridium thermocellum; the initial temperature is about 60° C.; and the initial pH is about 7.5.

In certain embodiments, the invention relates to a method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is Thermoanaerobacterium saccharolyticum; the second microorganism is Clostridium thermocellum; the ldh and pta genes of the Clostridium thermocellum have been partially, substantially, or completely deleted, silenced, inactivated, or down-regulated, thereby producing a genetically-modified Clostridium thermocellum; the initial temperature is about 60° C.; and the initial pH is about 7.5.

In certain embodiments, the invention relates to any one of the above-mentioned methods, further comprising the step of pretreating the lignocellulosic biomass. In certain embodiments, pretreating the lignocellulosic biomass comprises exposing the lignocellulosic biomass to steam autohydrolysis. In certain embodiments, pretreating the lignocellulosic biomass comprises milling the lignocellulosic biomass.

### Exemplary Microorganisms

The present invention includes multiple strategies for the use of co-cultures of microorganisms with the combination of substrate-utilization and product-formation properties required for CBP. For example, C. thermocellum is one of the best known cellulolytic anaerobes in nature; and T. saccharolyticum is a fermentative anaerobe with the capability to use the C5 sugars present in lignocellulosic biomass. The wild-type strains of these two microorganisms produce acetate, lactate, and ethanol as metabolic products. Utilizing co-cultures comprising these two microorganisms, in their wild-type or genetically engineered forms, for example, reduces the need for external enzymes to be added during the process.

Genetic engineering of microorganisms typically takes place in one of two fashions. The “native cellulolytic strategy” involves engineering naturally occurring cellulolytic microorganisms to improve product-related properties, such as yield and titer. The “recombinant cellulolytic strategy” involves engineering natively non-cellulolytic organisms that exhibit high product yields and titers to express a heterologous cellulase system that enables cellulose utilization or hemicellulose utilization or both.

In one aspect of the invention, the genes or particular polynucleotide sequences are inserted to activate the activity for which they encode, such as the expression of an enzyme. In certain embodiments, genes encoding enzymes in the metabolic production of ethanol, e.g., enzymes that metabolize pentose and/or hexose sugars, may be added to a mesophilic or thermophilic organism. In certain embodiments of the invention, the enzyme may confer the ability to metabolize a pentose sugar and be involved, for example, in the D-xylose pathway and/or L-arabinose pathway.

In one aspect of the invention, microorganisms are used in which one or more genes or particular polynucleotide sequences are partially, substantially, or completely deleted, silenced, inactivated, or down-regulated in order to inactivate the activity for which they encode, such as the expression of an enzyme. Deletions provide maximum stability because there is no opportunity for a reverse mutation to restore function. Alternatively, genes can be partially, substantially, or completely deleted, silenced, inactivated, or down-regulated by insertion of nucleic acid sequences that disrupt the function and/or expression of the gene (e.g., P1 transduction or other methods known in the art). The terms “eliminate,” “elimination,” and “knockout” are used interchangeably with the term “deletion.” In certain embodiments, strains of thermophilic or mesophilic microorganisms of interest may be engineered by site directed homologous recombination to knockout the production of organic acids. In still other embodiments, RNAi or antisense DNA (asDNA) may be used to partially, substantially, or completely silence, inactivate, or down-regulate a particular gene of interest.

In certain embodiments, the genes targeted for deletion or inactivation as described herein may be endogenous to the native strain of the microorganism, and may thus be understood to be referred to as “native gene(s)” or “endogenous gene(s).” An organism is in “a native state” if it has not been genetically engineered or otherwise manipulated by the hand of man in a manner that intentionally alters the genetic and/or phenotypic constitution of the organism. For example, wild-type organisms may be considered to be in a native state. In other embodiments, the gene(s) targeted for deletion or inactivation may be non-native to the organism.

Additionally, the pH or temperature tolerability of the microorganisms may be optimized to a certain degree. Certain microorganisms may be adapted to a certain temperature by selecting for a rapid growth rate over a period of time in a pH auxostat. Certain microorganisms may be adapted to a certain pH. This selection can be carried out by repeated batch transfers, that is, by transferring, for example, 1% inoculum to rich, undefined medium containing nutrients at successively higher pH over a period of time. By these methods, the temperature optimum or the pH optimum of a microorganism may be altered to better complement the temperature or pH optimum of another microorganism for use in a co-culture. In certain embodiments, pH-adapted strains of certain microorganisms can be successfully utilized in a co-culture where the wild-type of that microorganism did not grow well in the same co-culture.

Cellulolytic Microorganisms

Naturally occurring cellulolytic microorganisms are starting points for CBP organism development via the “native” strategy. Anaerobes and facultative anaerobes are of particular interest. The primary objective is to engineer product yields and ethanol titers to satisfy the requirements of an industrial process. Metabolic engineering of mixed-acid fermentations in relation to these objectives has been successful in the case of mesophilic, non-cellulolytic, enteric bacteria. Recent developments in suitable gene-transfer techniques allow for this type of work to be undertaken with cellulolytic bacteria.

Several microorganisms reported in the literature to be cellulolytic or have cellulolytic activity have been characterized by a variety of means, including their ability to grow on microcrystalline cellulose as well as a variety of other sugars. Additionally, the organisms may be characterized by other means, including but not limited to, their ability to depolymerize and debranch cellulose and hemicellulose. Clostridium thermocellum (strain DSMZ 1237) was used to benchmark the organisms of interest. As used herein, C. thermocellum may include various strains, including, but not limited to, DSMZ 1237, DSMZ 1313, DSMZ 2360, DSMZ 4150, DSMZ 7072, and ATCC 31924. In certain embodiments, the invention relates to a method utilizing a strain of C. thermocellum that may include, but is not limited to, DSMZ 1313 or DSMZ 1237. In certain embodiments, the invention relates to a method utilizing particularly suitable organisms of interest, including cellulolytic microorganisms with a greater than 70% 16S rDNA homology to C. thermocellum. Alignment of Clostridium thermocellum, Clostridium cellulolyticum, Thermoanaerobacterium saccharolyticum, C. stercorarium, C. stercorarium II, Caldiscellulosiruptor kristjanssonii, C. phytofermentans indicate a 73-85% homology at the level of the 16S rDNA gene.

Clostridium straminisolvens has been determined to grow nearly as well on Avicel® as does C. thermocellum. Table 1 summarizes certain highly cellulolytic organisms.

TABLE 1

| | | T | pH | | | | |
|---|---|---|---|---|---|---|---|
| | DSMZ | optimum; | optimum; | Gram | Aero- | | |
| Strain | No. | or range | or range | Stain | tolerant | Utilizes | Products |
| Clostridium | 1313 | 55-60 | 7 | positive | No | cellobiose, | acetic acid, |
| thermocellum | | | | | | cellulose | lactic acid, |
| | | | | | | | ethanol, H₂, |
| | | | | | | | CO₂ |
| Clostridium | 16021 | 50-55; | 6.5-6.8; | positive | Yes | cellobiose, | acetic acid, |
| straminisolvens | | 45-60 | 6.0-8.5 | | | cellulose | lactic acid, |
| | | | | | | | ethanol, H₂, |
| | | | | | | | CO₂ |
| Clostridium | 1237 | 55-60 | 7 | Positive | No | cellobiose, | acetic acid, |
| thermocellum | | | | | | cellulose | lactic acid, |
| | | | | | | | ethanol, H₂, |
| | | | | | | | CO₂ |

Certain microorganisms, including, for example, C. thermocellum and C. straminisolvens, cannot metabolize pentose sugars, such as D-xylose or L-arabinose, but are able to metabolize hexose sugars. Both D-xylose and L-arabinose are abundant sugars in biomass with D-xylose accounting for approximately 16-20% in soft and hard woods and L-arabinose accounting for approximately 25% in corn fiber. Accordingly, one object of the invention is to utilize genetically-modified cellulolytic microorganisms with the ability to metabolize pentose sugars, such as D-xylose and L-arabinose, thereby enhancing their use as biocatalysts for fermentation in the biomass-to-ethanol industry.

Cellulolytic and Xylanolytic Microorganisms

Several microorganisms determined from literature to be both cellulolytic and xylanolytic have been characterized by their ability to grow on microcrystalline cellulose and birchwood xylan as well as a variety of other sugars. Clostridium thermocellum was used to benchmark the organisms of interest. Of the strains selected for characterization Clostridium cellulolyticum, Clostridium stercorarium subs. leptospartum, Caldicellulosiruptor kristjanssonii and Clostridium phytofermentans grew weakly on Avicel® and well on birchwood xylan. Table 2 summarizes some of the native cellulolytic and xylanolytic organisms.

TABLE 2

| | | T | pH | | | | |
|---|---|---|---|---|---|---|---|
| | Source/ | optimum; | optimum; | Gram | Aero- | | |
| Strain | No. | or range | or range | Stain | tolerant | Utilizes | Products |
| Clostridium | DSM | 34 | 7.2 | negative | no | Cellulose, | acetic acid, |
| cellulolyticum | 5812 | | | | | xylan, | lactic acid, |
| | | | | | | arabinose, | ethanol, |
| | | | | | | mannose, | H₂, CO₂ |
| | | | | | | galactose, | |
| | | | | | | xylose, glucose, | |
| | | | | | | cellobiose | |
| Clostridium | DSM | 60-65 | 7.0-7.5 | negative | no | Cellulose, | acetic acid, |
| stercorarium subs. | 9219 | | | | | cellobiose, | lactic acid, |
| leptospartum | | | | | | lactose, xylose, | ethanol, |
| | | | | | | melibiose, | H₂, CO₂ |
| | | | | | | raffinose, | |
| | | | | | | ribose, fructose, | |
| | | | | | | sucrose | |
| Caldicellulosiruptor | DSM | 78; 45-82 | 7; 5.8-8.0 | negative | No | cellobiose, | acetic acid, |
| kristjanssonii | 12137 | | | | | glucose, xylose, | H₂, CO₂, |
| | | | | | | galactose, | lactic acid, |
| | | | | | | mannose, | ethanol |
| | | | | | | cellulose | formate |
| Clostridium | ATCC | 37; 5-45 | 8.5; 6-9 | Negative | no | Cellulose, | acetic acid, |
| phytofermentans | 700394 | | | (gram | | xylan, | H₂, CO₂, |
| | | | | type | | cellobiose, | lactic acid, |
| | | | | positive) | | fructose, | ethanol |
| | | | | | | galactose, | formate |
| | | | | | | glucose, lactose, | |
| | | | | | | maltose, | |
| | | | | | | mannose, | |
| | | | | | | ribose, xylose | |

Table 3 summarizes how bacterial strains may be categorized based on their substrate utilization.

TABLE 3

| | cellobiose | glucose | xylose | galactose | arabinose | mannose | lactose |
|---|---|---|---|---|---|---|---|
| C. cellulolyticum | x | x | x | x | x | | |
| C. stercorarium | x | x | x | x | x | x | x |
| subs. leptospartum | | | | | | | |
| C. kristjanssonii | x | x | x | x | | x | x |
| C. phytofermentans | x | x | x | x | | x | |

Non-Cellulolytic Microorganisms

Non-cellulolytic microorganisms with desired product-formation properties (e.g., high ethanol yield and titer) are starting points for CBP organism development by the recombinant cellulolytic strategy. The primary objective of such developments is to engineer a heterologous cellulase system that enables growth and fermentation on pretreated lignocellulose. The heterologous production of cellulases has been pursued primarily with bacterial hosts producing ethanol at high yield (engineered strains of E. coli, Klebsiella oxytoca, and Zymomonas mobilis) and the yeast Saccharomyces cerevisiae. Cellulase expression in strains of K. oxytoca resulted in increased hydrolysis yields—but not growth without added cellulase—for microcrystalline cellulose, and anaerobic growth on amorphous cellulose. Although dozens of saccharolytic enzymes have been functionally expressed in S. cerevisiae, anaerobic growth on cellulose as the result of such expression has not been definitively demonstrated.

Thermophilic and Mesophilic Microorganisms

Thermophilic or mesophilic cellulolytic microorganisms can be used as hosts for modification via the native cellulolytic strategy. Their potential in process applications in biotechnology, such as the methods of the present invention, stems from their ability to grow at relatively high temperatures with attendant high metabolic rates, production of physically and chemically stable enzymes, and elevated yields of end products. Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the invention relates to a method utilizing Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus. Some examples of thermophilic or mesophilic (including bacteria, procaryotic microorganism, and fungi), which may be suitable for use in the methods of the invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Clostridium phytofermentans, Clostridium straminosolvens, Thermoanaerobacterium thermosaccarolyticum, Thermoanaerobacterium saccharolyticum, Thermobacteroides acetoethylicus, Thermoanaerobium brockii, Methanobacterium thermoautotrophicum, Anaerocellum thermophilium, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus flavas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinate, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophile, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogens, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, variants thereof, or progeny thereof.

In certain embodiments, the invention relates to a method utilizing thermophilic bacteria selected from the group consisting of Fervidobacterium gondwanense, Clostridium thermolacticum, Moorella sp., and Rhodothermus marinus.

In certain embodiments, the invention relates to a method utilizing thermophilic bacteria of the genera Thermoanaerobacterium or Thermoanaerobacter, including, but not limited to, species selected from the group consisting of: Thermoanaerobacterium thermosulfurigenes, Thermoanaerobacterium aotearoense, Thermoanaerobacterium polysaccharolyticum, Thermoanaerobacterium zeae, xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In certain embodiments, the invention relates to a method utilizing microorganisms of the genera Geobacillus, Saccharococcus, Paenibacillus, Bacillus, and Anoxybacillus, including, but not limited to, species selected from the group consisting of: Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and progeny thereof.

In certain embodiments, the invention relates to a method utilizing mesophilic bacteria selected from the group consisting of Saccharophagus degradans; Flavobacterium johnsoniae; Fibrobacter succinogenes; Clostridium hungatei; Clostridium phytofermentans; Clostridium cellulolyticum; Clostridium aldrichii; Clostridium termitididis; Acetivibrio cellulolyticus; Acetivibrio ethanolgignens; Acetivibrio multivorans; Bacteroides cellulosolvens; and Alkalibacter saccharofomentans, variants thereof and progeny thereof.

Microorganisms for Use in Co-Cultures

In addition to any of the above-mentioned microorganisms, the following microorganisms may be used in a method of the present invention.

One or more of the microorganisms used in the methods of the present invention may be a wild-type thermophilic or mesophilic microorganism. In one embodiment, the invention relates to a method utilizing one or more wild-type thermophilic or mesophilic microorganisms, wherein said microorganism is a Gram-negative bacterium or a Gram-positive bacterium. In certain embodiments, the invention relates to a method utilizing any one or more of the above-mentioned wild-type microorganisms, wherein said wild-type microorganism is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus. In certain embodiments, the invention relates to a method utilizing any one or more of the above-mentioned wild-type microorganisms, wherein said wild-type microorganism is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellum thermophilum. In certain embodiments, the invention relates to a method utilizing any one or more of the above-mentioned wild-type microorganisms, wherein said wild-type microorganism is Thermoanaerobacterium saccharolyticum.

In certain embodiments, the invention relates to a method utilizing any one or more of the above-mentioned wild-type microorganisms, wherein said wild-type microorganism is selected from the group consisting of: (a) a thermophilic or mesophilic microorganism with a native ability to metabolize a hexose sugar; (b) a thermophilic or mesophilic microorganism with a native ability to metabolize a pentose sugar; and (c) a thermophilic or mesophilic microorganism with a native ability to metabolize a hexose sugar and a pentose sugar. In certain embodiments, the invention relates to a method utilizing any one or more of the above-mentioned wild-type microorganisms, wherein said wild-type microorganism has a native ability to metabolize a hexose sugar. In certain embodiments, the invention relates to a method utilizing any one or more of the above-mentioned wild-type microorganisms, wherein said wild-type microorganism is Clostridium straminisolvens or Clostridium thermocellum. In certain embodiments, the invention relates to a method utilizing any one or more of the above-mentioned wild-type microorganisms, wherein said wild-type microorganism is Clostridium thermocellum. In certain embodiments, the invention relates to a method utilizing any one or more of the above-mentioned wild-type microorganisms, wherein said wild-type microorganism has a native ability to metabolize a hexose sugar and a pentose sugar. In certain embodiments, the invention relates to a method utilizing any one or more of the above-mentioned wild-type microorganisms, wherein said wild-type microorganism is Clostridium cellulolyticum, Clostridium kristjanssonii, or Clostridium stercorarium subsp. leptosaprartum. In certain embodiments, the invention relates to a method utilizing any one or more of the above-mentioned wild-type microorganisms, wherein said wild-type microorganism has a native ability to metabolize a pentose sugar. In certain embodiments, the invention relates to a method utilizing any one or more of the above-mentioned wild-type microorganisms, wherein said wild-type microorganism is selected from the group consisting of Thermoanaerobacterium saccharolyticum, Thermoanaerobacterium xylanolyticum, Thermoanaerobacterium polysaccharolyticum, and Thermoanaerobacterium thermosaccharolyticum.

One or more microorganisms used in the methods of the invention may be a genetically-modified organism. These can be prepared by deleting or inactivating one or more genes that encode competing pathways, such as the non-limiting pathways to organic acids described herein, optionally followed by a growth-based selection for mutants with improved performance for producing ethanol as a fermentation product. In certain embodiments, the genetically-modified microorganisms used in the methods of the invention can be selected by a growth-based procedure to produce ethanol most efficiently at a certain initial temperature. In certain embodiments, the genetically-modified microorganisms used in the methods of the invention can be selected by a growth-based procedure to produce ethanol most efficiently at about 60° C. In certain embodiments, the genetically-modified microorganisms used in the methods of the invention can be selected by a growth-based procedure to produce ethanol most efficiently at a certain initial pH. In certain embodiments, the genetically-modified microorganisms used in the methods of the invention can be selected by a growth-based procedure to produce ethanol most efficiently at about pH 7.

In certain embodiments, gene knockout schemes can be applied individually or in concert to genetically-modified microorganisms used in the methods of the invention. Eliminating the mechanism for the production of lactate (i.e., knocking out the genes or particular polynucleotide sequences that encode for expression of LDH) generates more acetyl CoA; it follows that if the mechanism for the production of acetate is also eliminated (i.e., knocking out the genes or particular polynucleotide sequences that encode for expression of ACK or PTA), the abundance of acetyl CoA will be further enhanced, which should result in increased production of ethanol.

In certain embodiments, it is not required that the thermophilic or mesophilic microorganisms used in the methods of the invention have native or endogenous PDC or ADH. In certain embodiments, the genes encoding for PDC or ADH can be expressed recombinantly in the genetically-modified microorganisms used in the methods of the invention. In certain embodiments, gene knockout technology can be applied to recombinant microorganisms used in the methods of the invention, which recombinant microorganisms may comprise a heterologous gene that codes for PDC or ADH, wherein said heterologous gene is expressed at sufficient levels to increase the ability of said recombinant microorganism (which may be thermophilic) to produce ethanol as a fermentation product or to confer upon said recombinant microorganism (which may be thermophilic) the ability to produce ethanol as a fermentation product.

One or more of the microorganisms used in the methods of the invention may be genetically-modified thermophilic or mesophilic microorganisms, that is, the microorganisms may comprise at least one genetic modification. In one embodiment, the invention relates to a method utilizing one or more genetically-modified thermophilic or mesophilic microorganisms wherein a first native gene has been partially, substantially, or completely deleted, silenced, inactivated, or down-regulated, which first native gene encodes a first native enzyme involved in the metabolic production of an organic acid or a salt thereof, thereby increasing the native ability of said thermophilic or mesophilic microorganism to produce ethanol as a fermentation product. In certain embodiments, the invention relates to a method utilizing any one of the above-mentioned genetically-modified microorganisms, wherein said microorganism is a Gram-negative bacterium or a Gram-positive bacterium. In certain embodiments, the invention relates to a method utilizing any one of the above-mentioned genetically-modified microorganisms, wherein said microorganism is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus. In certain embodiments, the invention relates to a method utilizing any one of the above-mentioned genetically-modified microorganisms, wherein said microorganism is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellum thermophilum. In certain embodiments, the invention relates to a method utilizing any one of the above-mentioned genetically-modified microorganisms, wherein said microorganism is Thermoanaerobacterium saccharolyticum.

In certain embodiments, the invention relates to a method utilizing any one of the above-mentioned genetically-modified microorganisms, wherein said microorganism is selected from the group consisting of: (a) a thermophilic or mesophilic microorganism with a native ability to metabolize a hexose sugar; (b) a thermophilic or mesophilic microorganism with a native ability to metabolize a pentose sugar; and (c) a thermophilic or mesophilic microorganism with a native ability to metabolize a hexose sugar and a pentose sugar. In certain embodiments, the invention relates to a method utilizing any one of the above-mentioned genetically-modified microorganisms, wherein said microorganism has a native ability to metabolize a hexose sugar. In certain embodiments, the invention relates to a method utilizing any one of the above-mentioned genetically-modified microorganisms, wherein said microorganism is Clostridium straminisolvens or Clostridium thermocellum. In certain embodiments, the invention relates to a method utilizing any one of the above-mentioned genetically-modified microorganisms, wherein said microorganism has a native ability to metabolize a hexose sugar and a pentose sugar. In certain embodiments, the invention relates to a method utilizing any one of the above-mentioned genetically-modified microorganisms, wherein said microorganism is Clostridium cellulolyticum, Clostridium kristjanssonii, or Clostridium stercorarium subsp. leptosaprartum. In certain embodiments, the invention relates to a method utilizing any one of the above-mentioned genetically-modified microorganisms, wherein a first non-native gene has been inserted, which first non-native gene encodes a first non-native enzyme that confers the ability to metabolize a pentose sugar; thereby increasing the ability of said thermophilic or mesophilic microorganism to produce ethanol as a fermentation product from a pentose sugar. In certain embodiments, the invention relates to a method utilizing any one of the above-mentioned genetically-modified microorganisms, wherein said microorganism has a native ability to metabolize a pentose sugar. In certain embodiments, the invention relates to a method utilizing any one of the above-mentioned genetically-modified microorganisms, wherein said microorganism is selected from the group consisting of Thermoanaerobacterium saccharolyticum, Thermoanaerobacterium xylanolyticum, Thermoanaerobacterium polysaccharolyticum, and Thermoanaerobacterium thermosaccharolyticum. In certain embodiments, the invention relates to a method utilizing any one of the above-mentioned genetically-modified microorganisms, wherein a first non-native gene has been inserted, which first non-native gene encodes a first non-native enzyme that confers the ability to metabolize a hexose sugar; thereby increasing the ability of said thermophilic or mesophilic microorganism to produce ethanol as a fermentation product from a hexose sugar.

In certain embodiments, the invention relates to a method utilizing any one of the above-mentioned genetically-modified microorganisms, wherein said organic acid is selected from the group consisting of lactic acid and acetic acid. In certain embodiments, the invention relates to a method utilizing any one of the above-mentioned genetically-modified microorganisms, wherein said organic acid is lactic acid. In certain embodiments, the invention relates to a method utilizing any one of the above-mentioned genetically-modified microorganisms, wherein said organic acid is acetic acid. In certain embodiments, the invention relates to a method utilizing any one of the above-mentioned genetically-modified microorganisms, wherein said first native enzyme is selected from the group consisting of lactate dehydrogenase, acetate kinase, and phosphotransacetylase. In certain embodiments, the invention relates to a method utilizing any one of the above-mentioned genetically-modified microorganisms, wherein said first native enzyme is lactate dehydrogenase. In certain embodiments, the invention relates to a method utilizing any one of the above-mentioned genetically-modified microorganisms, wherein said first native enzyme is acetate kinase. In certain embodiments, the invention relates to a method utilizing any one of the above-mentioned genetically-modified microorganisms, wherein said first native enzyme is phosphotransacetylase.

In certain embodiments, the invention relates to a method utilizing any one of the above-mentioned genetically-modified microorganisms, wherein a second native gene has been partially, substantially, or completely deleted, silenced, inactivated, or down-regulated, which second native gene encodes a second native enzyme involved in the metabolic production of an organic acid or a salt thereof. In certain embodiments, the invention relates to a method utilizing any one of the above-mentioned genetically-modified microorganisms, wherein said second native enzyme is acetate kinase or phosphotransacetylase. In certain embodiments, the invention relates to a method utilizing any one of the above-mentioned genetically-modified microorganisms, wherein said second native enzyme is lactate dehydrogenase.

In one embodiment, the invention relates to a method utilizing any one or more of the genetically-modified thermophilic or mesophilic microorganisms, wherein (a) a first native gene has been partially, substantially, or completely deleted, silenced, inactivated, or down-regulated, which first native gene encodes a first native enzyme involved in the metabolic production of an organic acid or a salt thereof, and (b) a first non-native gene has been inserted, which first non-native gene encodes a first non-native enzyme involved in the metabolic production of ethanol; thereby increasing the ability of said thermophilic or mesophilic microorganism to produce ethanol as a fermentation product. In certain embodiments, the invention relates to a method utilizing any one of the above-mentioned genetically-modified microorganisms, wherein said first non-native gene encodes a first non-native enzyme that confers the ability to metabolize a hexose sugar, thereby allowing said thermophilic or mesophilic microorganism to metabolize a hexose sugar. In certain embodiments, the invention relates to a method utilizing any one of the above-mentioned genetically-modified microorganisms, wherein said first non-native gene encodes a first non-native enzyme that confers the ability to metabolize a pentose sugar, thereby allowing said thermophilic or mesophilic microorganism to metabolize a pentose sugar. In certain embodiments, the invention relates to a method utilizing any one of the above-mentioned genetically-modified microorganisms, wherein said first non-native gene encodes a first non-native enzyme that confers the ability to metabolize a hexose sugar; and a second non-native gene is inserted, which second non-native gene encodes a second non-native enzyme that confers the ability to metabolize a pentose sugar, thereby allowing said thermophilic or mesophilic microorganism to metabolize a hexose sugar and a pentose sugar.

In certain embodiments, the invention relates to a method utilizing any one of the above-mentioned genetically-modified microorganisms, wherein said organic acid is lactic acid. In certain embodiments, the invention relates to a method utilizing any one of the above-mentioned genetically-modified microorganisms, wherein said organic acid is acetic acid.

In certain embodiments, the invention relates to a method utilizing any one of the above-mentioned genetically-modified microorganisms, wherein said first non-native enzyme is pyruvate decarboxylase (PDC) or alcohol dehydrogenase (ADH). In certain embodiments, the invention relates to a method utilizing any one of the above-mentioned genetically-modified microorganisms, wherein said second non-native enzyme is xylose isomerase. In certain embodiments, the invention relates to a method utilizing any one of the above-mentioned genetically-modified microorganisms, wherein said non-native enzyme is xylulokinase. In certain embodiments, the invention relates to a method utilizing any one of the above-mentioned genetically-modified microorganisms, wherein said non-native enzyme is L-arabinose isomerase. In certain embodiments, the invention relates to a method utilizing any one of the above-mentioned genetically-modified microorganisms, wherein said non-native enzyme is L-ribulose-5-phosphate 4-epimerase.

In certain embodiments, the invention relates to a method utilizing any one of the above-mentioned genetically-modified microorganisms, wherein said microorganism is selected from the group consisting of: (a) a thermophilic or mesophilic microorganism with a native ability to hydrolyze cellulose; (b) a thermophilic or mesophilic microorganism with a native ability to hydrolyze xylan; and (c) a thermophilic or mesophilic microorganism with a native ability to hydrolyze cellulose and xylan.

In certain embodiments, the invention relates to a method utilizing any one of the above-mentioned genetically-modified microorganisms, wherein said microorganism has a native ability to hydrolyze cellulose. In certain embodiments, the invention relates to a method utilizing any one of the above-mentioned genetically-modified microorganisms, wherein said microorganism has a native ability to hydrolyze cellulose and xylan. In certain embodiments, the invention relates to a method utilizing any one of the above-mentioned genetically-modified microorganisms, wherein a first non-native gene is inserted, which first non-native gene encodes a first non-native enzyme that confers the ability to hydrolyze xylan.

In certain embodiments, the invention relates to a method utilizing any one of the above-mentioned genetically-modified microorganisms, wherein said microorganism has a native ability to hydrolyze xylan. In certain embodiments, the invention relates to a method utilizing any one of the above-mentioned genetically-modified microorganisms, wherein a first non-native gene has been inserted, which first non-native gene encodes a first non-native enzyme that confers the ability to hydrolyze cellulose.

In certain embodiments, the invention relates to a method utilizing any one of the above-mentioned genetically-modified microorganisms, wherein said organic acid is selected from the group consisting of lactic acid and acetic acid. In certain embodiments, the invention relates to a method utilizing any one of the above-mentioned genetically-modified microorganisms, wherein said organic acid is lactic acid. In certain embodiments, the invention relates to a method utilizing any one of the above-mentioned genetically-modified microorganisms, wherein said organic acid is acetic acid.

In certain embodiments, the invention relates to a method utilizing any one of the above-mentioned genetically-modified microorganisms, wherein said first native enzyme is selected from the group consisting of lactate dehydrogenase, acetate kinase, and phosphotransacetylase. In certain embodiments, the invention relates to a method utilizing any one of the above-mentioned genetically-modified microorganisms, wherein said first native enzyme is lactate dehydrogenase. In certain embodiments, the invention relates to a method utilizing any one of the above-mentioned genetically-modified microorganisms, wherein said first native enzyme is acetate kinase. In certain embodiments, the invention relates to a method utilizing any one of the above-mentioned genetically-modified microorganisms, wherein said first native enzyme is phosphotransacetylase.

In certain embodiments, the invention relates to a method utilizing any one of the above-mentioned genetically-modified microorganisms, wherein a second native gene has been partially, substantially, or completely deleted, silenced, inactivated, or down-regulated, which second native gene encodes a second native enzyme involved in the metabolic production of an organic acid or a salt thereof. In certain embodiments, the invention relates to a method utilizing any one of the above-mentioned genetically-modified microorganisms, wherein said second native enzyme is acetate kinase or phosphotransacetylase. In certain embodiments, the invention relates to a method utilizing any one of the above-mentioned genetically-modified microorganisms, wherein said second native enzyme is lactate dehydrogenase.

In one embodiment, the invention relates to a method utilizing one or more genetically-modified microorganisms comprising (a) a first native gene that has been partially, substantially, or completely deleted, silenced, inactivated, or down-regulated, which first native gene encodes a first native enzyme involved in the metabolic production of an organic acid or a salt thereof, and (b) a first non-native gene that has been inserted, which first non-native gene encodes a first non-native enzyme involved in the hydrolysis of a polysaccharide; thereby increasing the ability of said thermophilic or mesophilic microorganism to produce ethanol as a fermentation product. In certain embodiments, the invention relates to a method utilizing any one of the above-mentioned genetically-modified microorganisms, wherein said first non-native gene encodes a first non-native enzyme that confers the ability to hydrolyze cellulose, thereby allowing said thermophilic or mesophilic microorganism to hydrolyze cellulose. In certain embodiments, the invention relates to a method utilizing any one of the above-mentioned genetically-modified microorganisms, wherein said first non-native gene encodes a first non-native enzyme that confers the ability to hydrolyze xylan, thereby allowing said thermophilic or mesophilic microorganism to hydrolyze xylan. In certain embodiments, the invention relates to a method utilizing any one of the above-mentioned genetically-modified microorganisms, wherein said first non-native gene encodes a first non-native enzyme that confers the ability to hydrolyze cellulose; and a second non-native gene has been inserted, which second non-native gene encodes a second non-native enzyme that confers the ability to hydrolyze xylan, thereby allowing said thermophilic or mesophilic microorganism to hydrolyze cellulose and xylan.

In certain embodiments, the invention relates to a method utilizing any one of the above-mentioned genetically-modified microorganisms, wherein said organic acid is lactic acid. In certain embodiments, the invention relates to a method utilizing any one of the above-mentioned genetically-modified microorganisms, wherein said organic acid is acetic acid.

In certain embodiments, the invention relates to a method utilizing any one of the above-mentioned genetically-modified microorganisms, wherein said first non-native enzyme is pyruvate decarboxylase (PDC) or alcohol dehydrogenase (ADH).

In certain embodiments, the invention relates to a method utilizing any one of the above-mentioned genetically-modified microorganisms, wherein said microorganism is mesophilic. In certain embodiments, the invention relates to a method utilizing any one of the above-mentioned genetically-modified microorganisms, wherein said microorganism is thermophilic.

In certain embodiments, the invention relates to a method utilizing any one of the above-mentioned genetically-modified microorganisms, wherein said microorganism is Thermoanaerobacterium saccharolyticum; and a mutant of said microorganism has been selected by a growth-based procedure to produce ethanol most efficiently at a specific temperature. In certain embodiments, the invention relates to a method utilizing any one of the above-mentioned genetically-modified microorganisms, wherein said microorganism is Thermoanaerobacterium saccharolyticum; and a mutant of said microorganism has been selected by a growth-based procedure to produce ethanol most efficiently at about 60° C.

In certain embodiments, the invention relates to a method utilizing any one of the above-mentioned genetically-modified microorganisms, wherein said microorganism is Thermoanaerobacterium saccharolyticum; and a mutant of said microorganism has been selected by a growth-based procedure to produce ethanol most efficiently at a specific pH. In certain embodiments, the invention relates to a method utilizing any one of the above-mentioned genetically-modified microorganisms, wherein said microorganism is Thermoanaerobacterium saccharolyticum; and a mutant of said microorganism has been selected by a growth-based procedure to produce ethanol most efficiently at about pH 7.

In certain embodiments, the invention relates to a method utilizing any one of the above-mentioned genetically-modified microorganisms, wherein said microorganism is Thermoanaerobacterium saccharolyticum; and a mutant of said microorganism has been selected by a growth-based procedure to produce ethanol most efficiently at a specific temperature and a specific pH. In certain embodiments, the invention relates to a method utilizing any one of the above-mentioned genetically-modified microorganisms, wherein said microorganism is Thermoanaerobacterium saccharolyticum; and a mutant of said microorganism has been selected by a growth-based procedure to produce ethanol most efficiently at about 60° C. and about pH 7.

In certain embodiments, the invention relates to a method utilizing any one of the above-mentioned genetically-modified microorganisms, wherein said microorganism has been selected for tolerability at a certain temperature. In certain embodiments, said microorganism was adapted to a rapid growth rate at a temperature in a pH auxostat for a period of time. In certain embodiments, said temperature is about 60° C. In certain embodiments, said period of time is about three months.

In certain embodiments, the invention relates to a method utilizing any one of the above-mentioned genetically-modified microorganisms, wherein said microorganism has been adapted to a certain pH over a period of time. In certain embodiments, said pH adaptation was carried out by transferring 1% inoculum to rich, undefined medium containing nutrients at successively higher pH. In certain embodiments, said nutrients are selected from the group consisting of xylose, glucose, or cellobiose. In certain embodiments, said rich, undefined medium is MTC. In certain embodiments, said microorganisms begin at pH 5.8. In certain embodiments, said microorganisms are transferred twice to medium at pH 6.3. In certain embodiments, said microorganisms are transferred three times to medium at pH 6.6. In certain embodiments, said microorganisms are transferred seven times to medium at pH 7.0. In certain embodiments, said pH adaptation allows said microorganism to grow in a co-culture with one or more other microorganisms, wherein said microorganism did not grow in a co-culture with said one or more other microorganisms prior to said pH adaptation.

In certain embodiments, the invention relates to a method utilizing any one of the above-mentioned genetically-modified microorganisms, wherein said microorganism has been selected for tolerability at a certain temperature and adapted to a certain pH. In certain embodiments, said microorganism was adapted to a rapid growth rate at a temperature in a pH auxostat for a period of time. In certain embodiments, said temperature is about 60° C. In certain embodiments, said period of time is about three months. In certain embodiments, said pH adaptation was carried out by transferring 1% inoculum to rich, undefined medium containing nutrients at successively higher pH. In certain embodiments, said nutrients are selected from the group consisting of xylose, glucose, or cellobiose. In certain embodiments, said rich, undefined medium is MTC. In certain embodiments, said microorganisms begin at pH 5.8. In certain embodiments, said microorganisms are transferred twice to medium at pH 6.3. In certain embodiments, said microorganisms are transferred three times to medium at pH 6.6. In certain embodiments, said microorganisms are transferred seven times to medium at pH 7.0. In certain embodiments, said temperature selection occurs prior to said pH adaptation.

### Exemplary Lignocellulosic Biomass

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; and forestry wastes, such as but not limited to recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Particularly advantageous lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Paper sludge is also a viable feedstock for ethanol production. Paper sludge is solid residue arising from pulping and paper-making, and is typically removed from process wastewater in a primary clarifier. At a disposal cost of $30/wet ton, the cost of sludge disposal equates to $5/ton of paper that is produced for sale. The cost of disposing of wet sludge is a significant incentive to convert the material for other uses, such as conversion to ethanol. Methods provided by the present invention are widely applicable. Moreover, the saccharification and/or fermentation products may be used to produce ethanol or higher value added chemicals, such as organic acids, aromatics, esters, acetone and polymer intermediates.

In one embodiment, the present invention relates to methods for converting lignocellulosic biomass into ethanol, wherein said lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof. In certain embodiments, the present invention relates to the above-mentioned method, wherein said lignocellulosic biomass is selected from the group consisting of corn stover, sugarcane bagasse, switchgrass, and poplar wood. In certain embodiments, the present invention relates to the above-mentioned method, wherein said lignocellulosic biomass is corn stover. In certain embodiments, the present invention relates to the above-mentioned method, wherein said lignocellulosic biomass is sugarcane bagasse. In certain embodiments, the present invention relates to the above-mentioned method, wherein said lignocellulosic biomass is switchgrass. In certain embodiments, the present invention relates to the above-mentioned method, wherein said lignocellulosic biomass is poplar wood. In certain embodiments, the present invention relates to the above-mentioned method, wherein said lignocellulosic biomass is willow. In certain embodiments, the present invention relates to the above-mentioned method, wherein said lignocellulosic biomass is paper sludge.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

Fermentations were performed in 125-mL serum bottles with modified CTFUD medium (Table 4). The substrate used was hardwood pretreated by steam autohydrolysis (MS149) which was thoroughly washed, dried and milled to pass through a 0.5-mm screen. The bottles containing a monoculture of C. thermocellum contained 40 mL of medium, and the co-culture bottles contained 35 mL of medium. Each bottle was inoculated with 10 mL of C. thermocellum actively growing on 1% Avicel. The T. saccharolyticum strain used was an engineered ldh-, ack-construct subsequently adapted to grow at 60° C. and pH 7, through the use of repeated batch transfers (see details below). In the co-culture, 5 mL of cellobiose grown engineered T. saccharolyticum were then used for inoculation. Fermentations were performed in a shaker incubator at 60° C., at a shaking speed of 125 rpm. The initial pH in the serum bottles was 7.0.

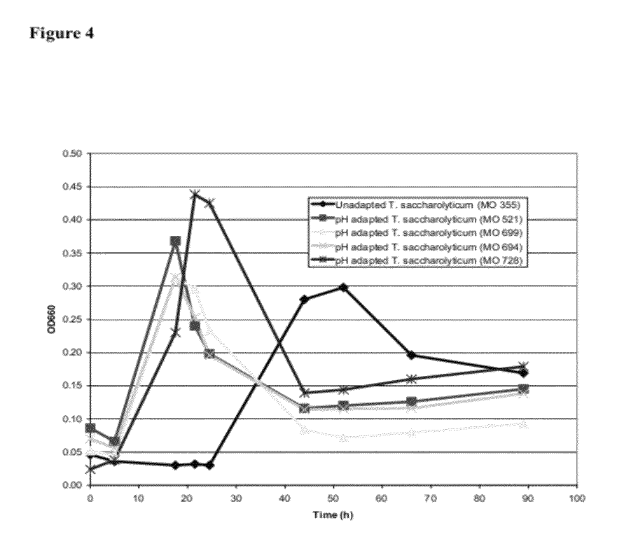

The T. saccharolyticum strain used was engineered in three ways. First, phosphotransacetylase/acetate kinase and lactate dehydrogenase genes were knocked out to eliminate lactic acid and acetic acid production during fermentation (MO 355). Second, MO 355 was adapted to a rapid growth rate (μ=0.27 h⁻¹) over three months in a pH auxostat. Lastly, this strain was adapted to pH 7 by serial transfer in tube culture. The high pH adaptation was carried out over six days by transferring 1% inoculum to rich, undefined medium (MTC) containing 5 g/L each of xylose, glucose, and cellobiose at successively higher pH. Beginning at pH 5.8, the strain was transferred twice to a medium at pH 6.3, then three times to a medium at pH 6.6, then seven times to a medium at 7.0 before being stored at −80° C. for future experiments (MO 728). The optimal pH for wild-type T. saccharolyticum is 6.0, while that of wild-type C. thermocellum is 7.0. Evidence that the pH adaptation method used with T. saccharolyticum was successful is shown in FIG. 4. When four strains that were adapted to pH 7.0 were transferred from frozen stocks into CTFUD medium at pH 7.0, each strain exhibited a lag phase reduced by approximately 20 h compared to the unadapted parent strain that prefers growth at pH 6.0.

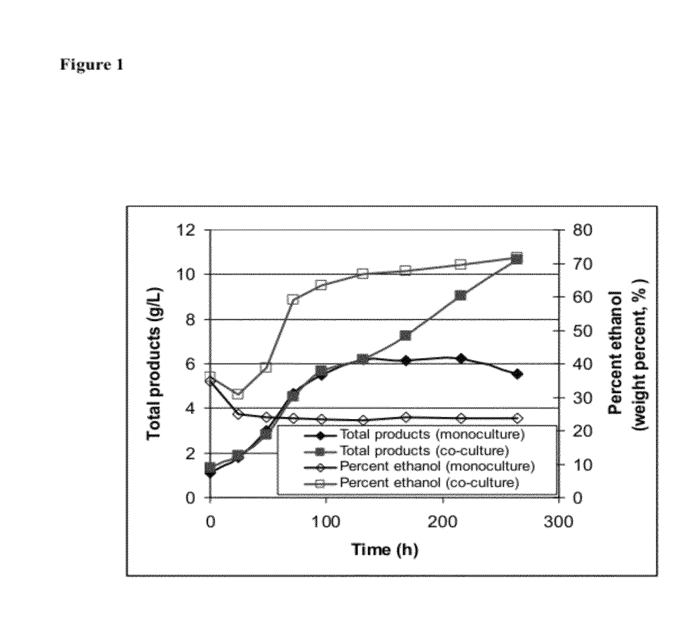

The fermentation profiles for the monoculture and co-culture are shown in FIG. 1. The total product concentrations in the monoculture and co-culture were similar up to 120 h. The percent of ethanol in the co-culture (g ethanol/g total products) was about 70% compared to 25% in the monoculture. After 120 h of fermentation, the total product concentration of the co-culture was higher than the monoculture. There was no further product accumulation in the monoculture fermentations after 120 h of fermentation. The final pH in the monoculture was about 6.1 compared to 5.7 for the co-culture. Although the cessation of fermentation could be attributed to low pH, the results indicate the co-culture is more tolerant to low pH than C. thermocellum alone.

The carbohydrate content of the thoroughly washed pretreated mixed hardwood was 55.6% Glucan and 4.21% Xylan. The percent product yield (percent of total initial carbohydrate (glucan+xylan) used in producing ethanol, acetate, and lactate) at the end of fermentation was 23% in a monoculture compared to 56% in a co-culture. The percent total soluble products yield (percent of total initial carbohydrate (glucan+xylan) used in producing glucose, cellobiose, xylose, ethanol, acetate, and lactate) was 30% in the monoculture compared to 56% in the co-culture. The accumulation of soluble sugars (glucose, xylose, and cellobiose) in the monoculture accounted for the 7% difference between the two yield calculations in the monoculture. There was no accumulation of soluble sugars in the co-culture, so the two yield values are the same. The percent ethanol yield based on initial carbohydrate (percent of total initial carbohydrate used in producing ethanol) is 7.7% in monoculture compared to 44.5% in co-culture.

TABLE 4
Medium composition

| | Modified |
|---|---|
| Components | CTFUD (g/L) |
| Phosphates | |
| KH₂PO₄ | 1.43 |
| K₂HPO₄ | 1.8 |
| Total | 3.23 |
| Nitrogen | |
| (NH₄)₂SO₄ | 2.6 |
| Yeast extract | 9.0 |
| Total | 11.6 |
| Metals | |
| CaCl₂ | 0.13 |
| MgCl₂ | 2.6 |
| Total | 2.73 |
| Reductants | |
| Cysteine HCl | 0.5 |
| Buffer | |
| Sodium bicarbonate | 5 |
| Sodium citrate tribasic dehydrate | 3 |
| MOPS | 10 |
| Total | 13 |

### Example 2

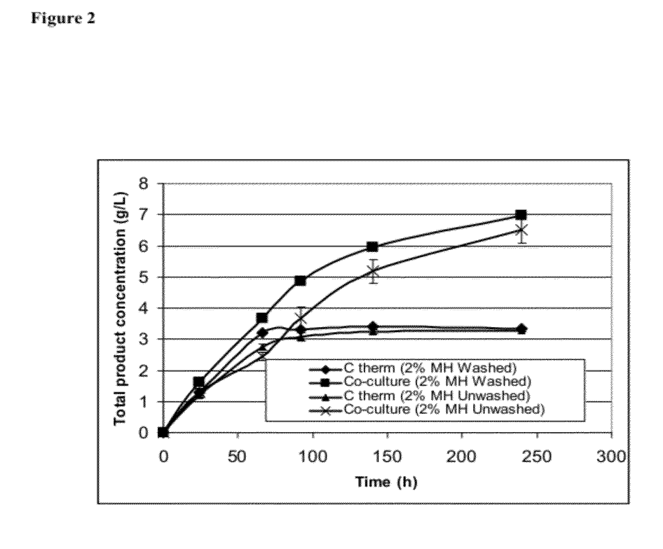

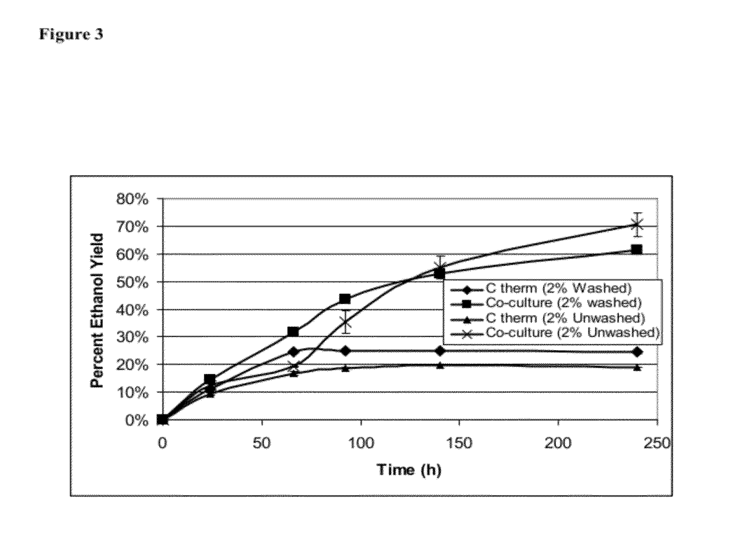

An experiment similar to that performed in Example 1 was performed using hardwood pretreated by steam autohydrolysis, washed or unwashed, dried and milled to pass through a 0.5-mm screen. The bottles containing a monoculture of C. thermocellum contained 40 mL of medium, and the co-culture bottles contained 35 mL of medium. The medium used is the same medium as shown in Table 4. The total product concentration (FIG. 2) was about 7 g/L on washed and unwashed mixed hardwood compared to 3.4 g/L in C. thermocellum monoculture. The total product concentration in the co-culture was about double the total product concentration in the C. thermocellum monoculture, as in Example 1. The percent ethanol yield based on carbohydrates present in the solid fraction is shown in FIG. 3. The percent ethanol yield on washed and unwashed mixed hardwoods was above 60% for a co-culture on 2% mixed hardwoods. The percent ethanol yield in the monoculture, on the other hand, was less than 25% on the same 2% mixed hardwood substrate.

### Example 3

### Co-culture fermentation data using LDH KO C. thermocellum and T. saccharolyticum

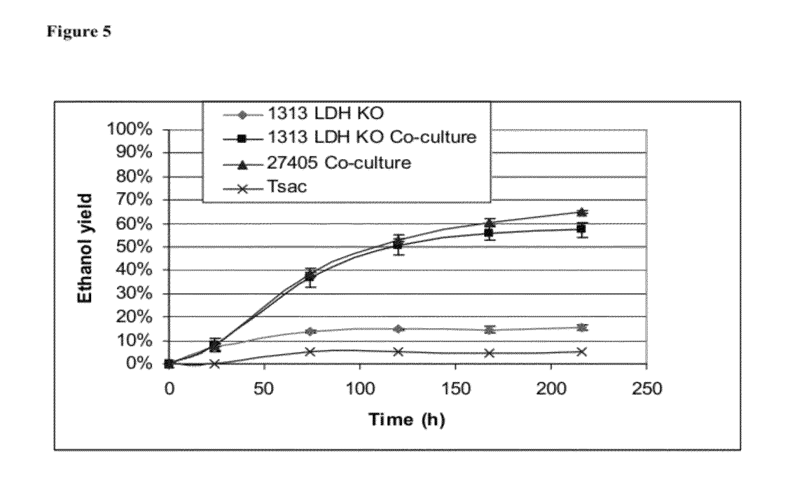

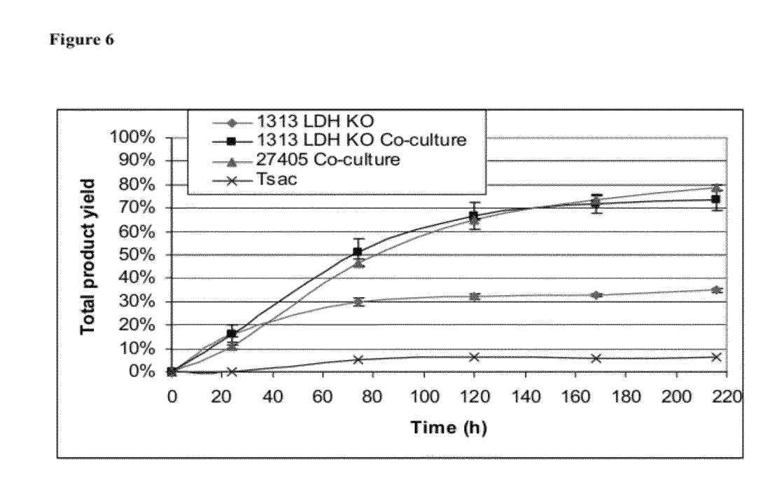

This experiment was performed to determine the product concentrations that can be obtained in a co-culture on 2% washed mixed hardwoods using two different strains of C. thermocellum. The wild type 27405 and the LDH-KO 1313 strain were used for this study. Fermentations were started at a temperature of 60° C. and an initial pH of 7.5 in serum bottles using the medium in Table 4. The ethanol yields are shown in FIG. 5. The data shows that an ethanol yield of 60% could be achieved by both strains. The total product yield, FIG. 6 (accounting for acetate and lactate) was above 73% of the theoretical yield for both strains in 220 h.

### Example 4

### Co-Culture Data on the Residue from Yeast Fermentation

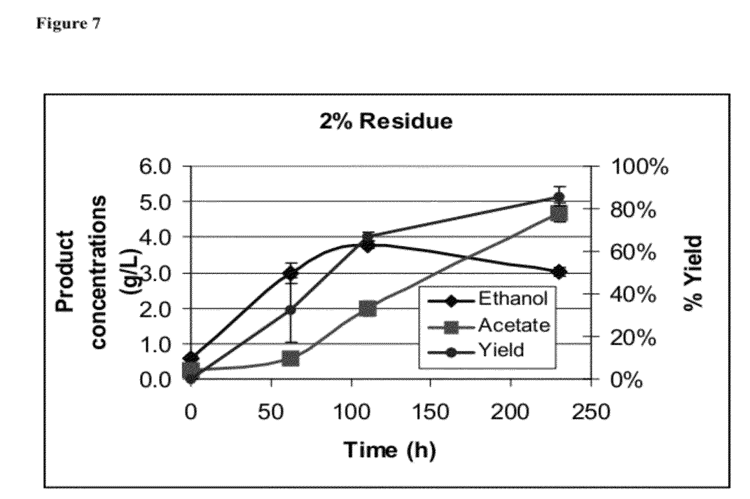

The residue remaining after yeast fermentation was used as a substrate for co-culture fermentation. The residue was washed a couple of times to remove soluble products such as ethanol and dried at 40° C. The dried material was used as a substrate in a co-culture fermentation using C. thermocellum and T. saccharolyticum. The total carbohydrate yield of 80% (FIG. 7) was achieved and accounting for the 60% from yeast fermentation, this results in a total carbohydrate yield of about 90%.

### Example 5

### Fermentation Data on 80 g/L Avicel Fermentation

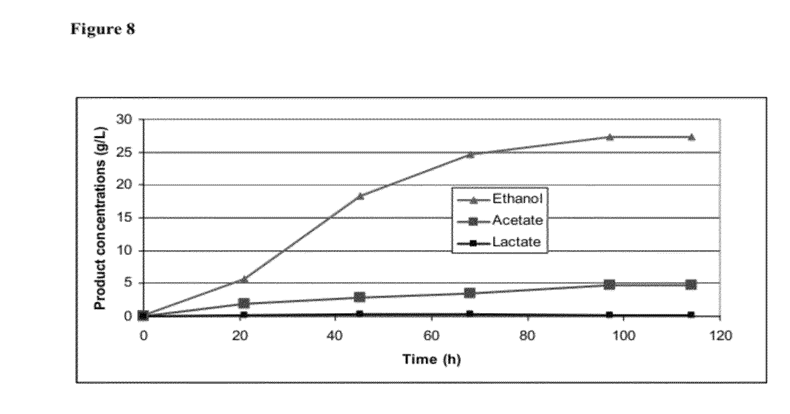

In this experiment, co-culture fermentation was performed using an initial Avicel concentration of 80 g/L in a bioreactor. The co-culture fermentation was performed by using MTC medium. Fermentations were performed at 55° C. and a pH 6. The fermentation profile is shown in FIG. 8. The maximum ethanol concentration was 27 g/L and this was achieved in about 97 h. The theoretical ethanol yield was 60% and the yield based on other by-products (acetate+lactate) was 68%.

### Example 6

### Co-Culture Data on 180 g/L Avicel Fermentation

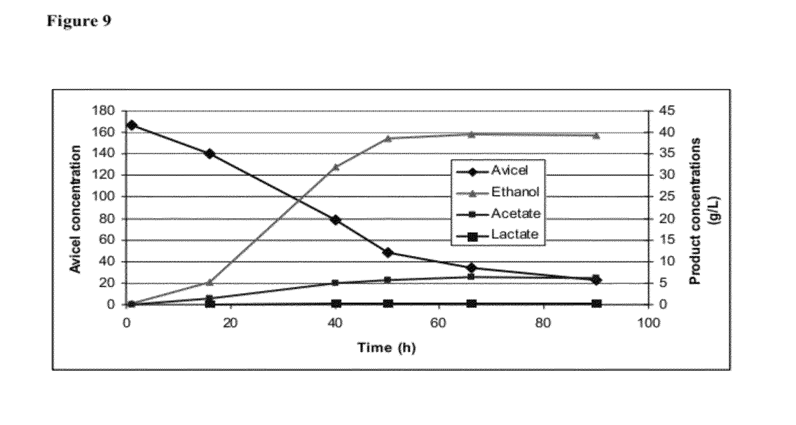

In this experiment, co-culture fermentation was performed using an initial Avicel concentration of 160 g/L in a bioreactor. The co-culture fermentation was performed by using MTC medium with additional components. The other components that were added to MTC was; 5 g/L CaCO₃, 5 g/L MgCO₃, 5 g/L yeast extract, 0.3 g/L methionine, 1 g/L Resazurin and 1 g/L Cysteine HCl. Fermentations were performed at 55° C. and an pH 6.3. The fermentation profile is shown in FIG. 9. The rate of cellulose utilization in the co-culture (2.7 g/Lh) is higher than the rate of cellulose utilization reported for C. thermocellum monoculture (˜1.2 g/Lh). The maximum ethanol concentration was 40 g/L and this was achieved in about 40 h. The theoretical ethanol yield was 58% and the yield based on other by-products (acetate+lactate) was 65%.

### Example 7

### Fermentation Data at Various Avicel Concentrations (40, 80 and 120 g/L)

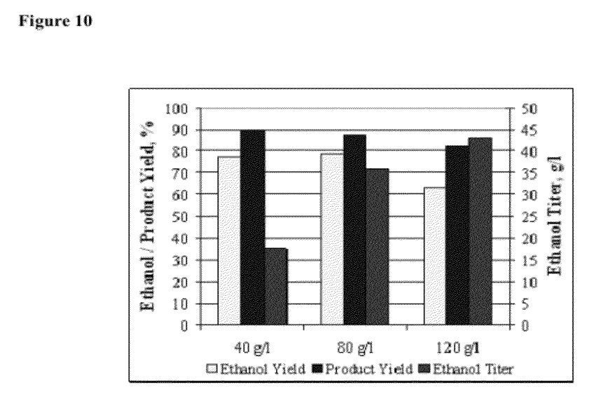

Co-culture fermentations were performed on Avicel at concentrations of 40 g/L, 80 g/L and 120 g/L. T. saccharolyticum (10% inoculation) and C. thermocellum (10% inoculation) were used as inoculums for the fermentations. The medium used was MTC+5 g/L CaCO₃. The yields and the titers are shown in FIG. 10. The data showed that an ethanol yield in the range 65-75% could be attained and a total product yield of 80-90% could be achieved.

### Example 8

### Fermentation Data on Unwashed Mixed Hardwood (MS149) Using Milled and Unmilled Samples

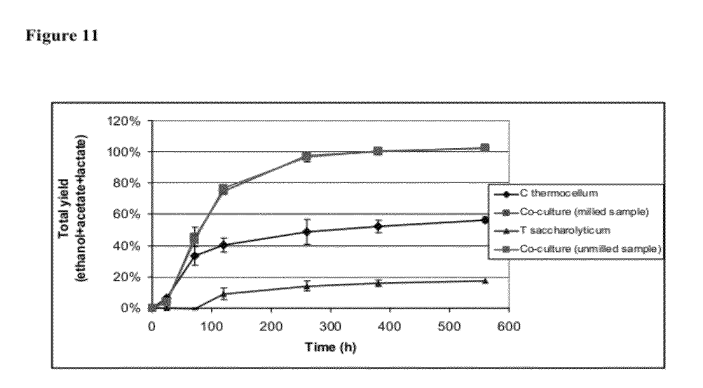

An experiment was performed using hardwood pretreated by steam autohydrolysis unwashed, dried and milled to pass through a 0.5-mm screen. Unmilled samples were used as a control. The medium used is the same medium as shown in Table 4. The potential total product yield (ethanol+acetate+lactate) based total available carbohydrates are shown in FIG. 11. Fermentations were started at a temperature of 60° C. and an initial pH of 7.5 in serum bottles.

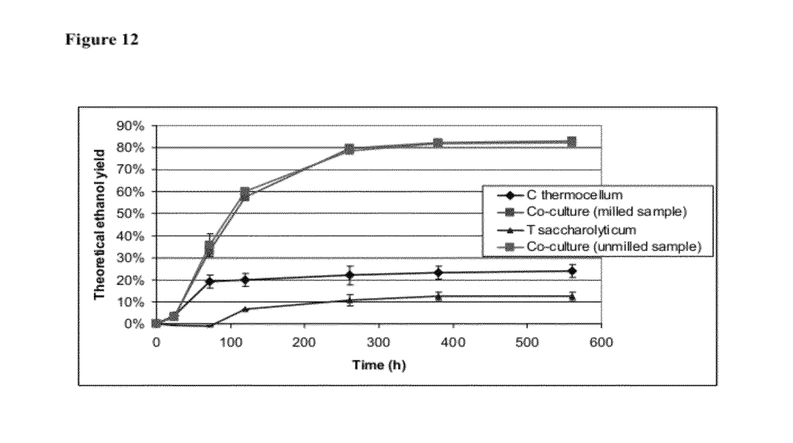

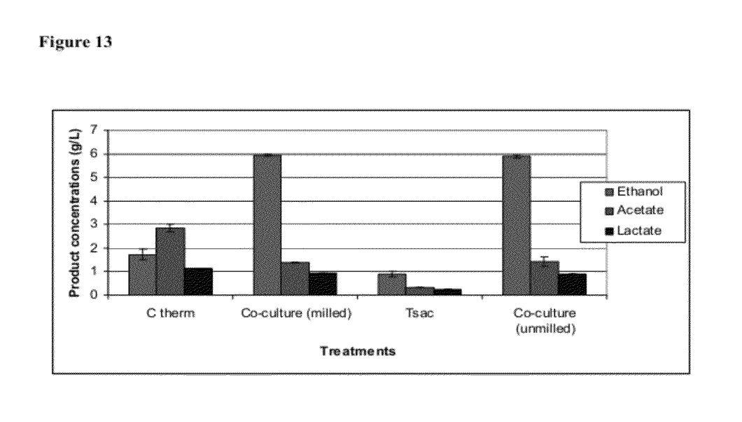

The data show that, whereas T. saccharolyticum monoculture attained less than 20% of the total product yield, C. thermocellum achieved close to 60% of the total product yield. Co-cultures of the two organisms achieved close to 100% theoretical yield with milled or unmilled samples. Milling does not appear to affect the hydrolysis rate or final total product yield. The theoretical ethanol yields for each of the treatments are shown in FIG. 12. The theoretical ethanol yield was slightly higher than 10% for T. saccharolyticum, slightly above 20% for C. thermocellum and about 80% for co-cultures with milled and unmilled samples. FIG. 13 shows the product distributions obtained. Whereas acetate was the major product with the C. thermocellum fermentation, ethanol was the major product in the co-cultures and T. saccharolyticum controls.

### Example 9

### Co-Culture Fermentation Data on 2-7.5% Solids on Mixed Hardwoods

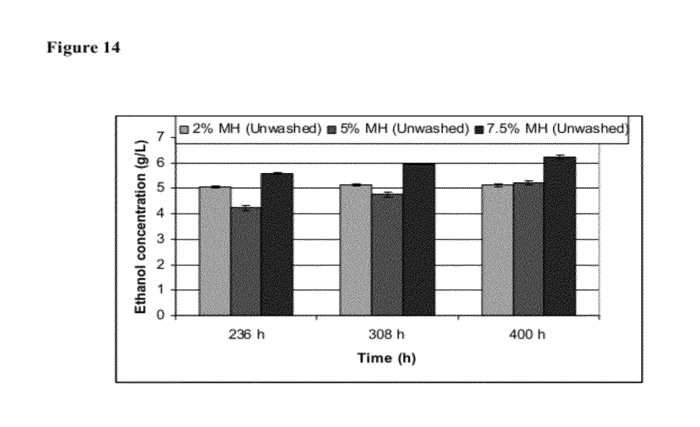

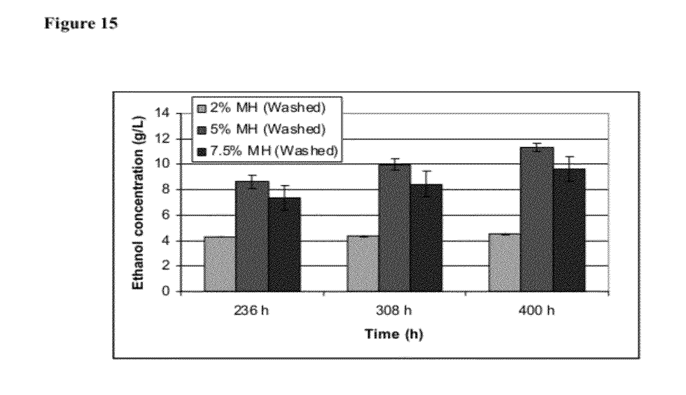

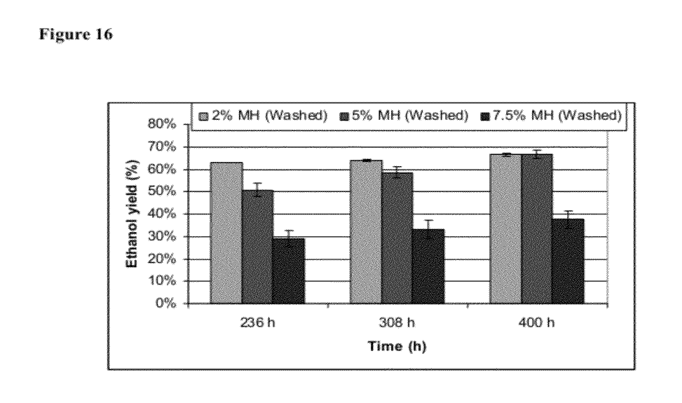

This experiment was performed to determine the product concentrations that can be obtained in a co-culture at 5-7.5% solids loading on washed and unwashed material. Fermentations were started at a temperature of 60° C. and an initial pH of 7.5 in serum bottles using the medium in Table 4. The ethanol concentration at the various solids loadings on unwashed materials are shown in FIG. 14. In the fermentations containing 2 and 5% unwashed materials, 5 g/L of ethanol was obtained in 400 h whereas the maximum ethanol concentration obtained on 7.5% solids was 6 g/L. The ethanol concentrations obtained on extensively washed material is shown in FIG. 15. The data show that about 10 g/L ethanol could be achieved on 5 and 7.5% washed substrate. The theoretical ethanol yield from these fermentations is shown in FIG. 16. From FIG. 16, a theoretical ethanol yield of more the 65% was attained on 2% and 5% washed materials. Accounting for other products, such as acetate and lactate, the total product yield on 2% washed material was 90% and the yield on 5% solids was 80% of theoretical yield

### Example 10

### Co-Culture Data Extensively Washed Mixed Hardwoods

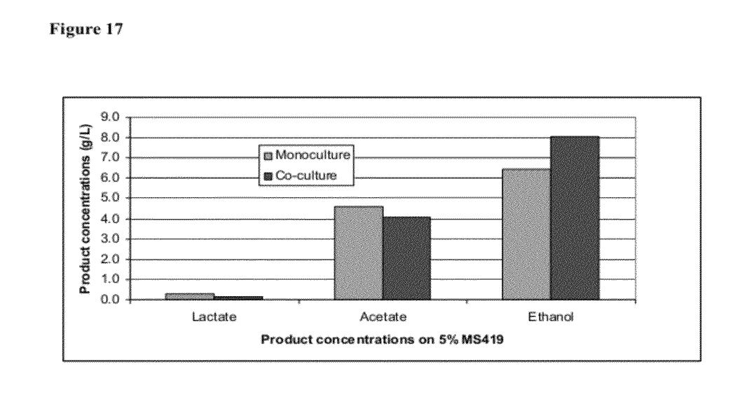

In this experiment, monoculture and co-culture fermentations were performed on extensively washed mixed hardwood. The co-culture fermentation was performed by using MTC medium with additional components. The other components that were added to MTC were: 5 g/L yeast extract, 5 g/L CaCO₃, 1 g/L Resazurin and 1 g/L Cysteine HCl. Fermentations were performed at 55° C. and an pH 6.3. The final product concentration after 165 h of fermentation can be shown in FIG. 17. The theoretical ethanol yield was 38% for the monoculture and 48% for the co-culture. The total product yield was 60% for the monoculture and 66% for the co-culture.

### Example 11

### Fermentation Data on Paper Sludge

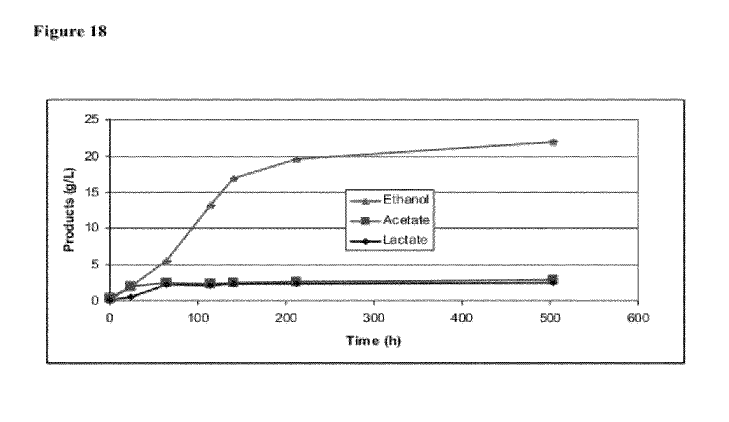

Fermentations were started at a temperature of 60° C. and an initial pH of 7.5 in serum bottles. The product concentrations obtained are shown in FIG. 18. Ethanol was the major product and a final ethanol concentration of 22 g/L was obtained on 10% paper sludge. Fermentations were started at a temperature of 60° C. and an initial pH of 7.5 in serum bottles. The theoretical ethanol yield was 62.4% and the total product yield (percent of total initial carbohydrates converted to ethanol, acetate and lactate) of 72.4% was obtained on 100 g/L paper sludge. The paper sludge used for this fermentation contains 48.2% cellulose and 13.9% xylan, which corresponds to a yield of 108 gal/dry ton of feedstock. From the yield values, an ethanol yield of 67 gal/dry ton and a total product yield of 78 gal/dry ton could be obtained if the acetate and lactate were converted to ethanol.

### Example 12

### Fermentation Data on a Mixture of 3% Paper Sludge and 15% Unwashed Mixed Hardwood

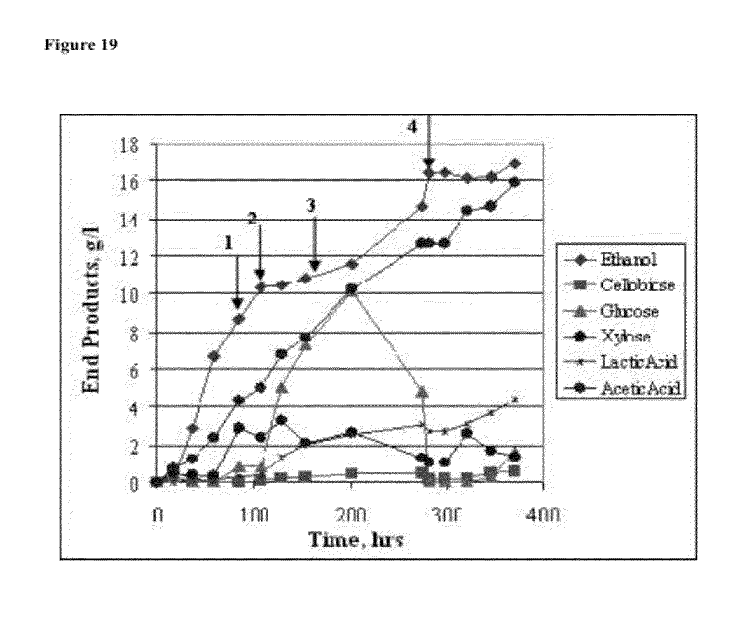

Co-culture of unwashed mixed hardwoods, 3% paper sludge plus 2% of unwashed mixed hardwood was used to start the culture at time zero. More mixed hardwood was fed into the fermentation until the solids reached 14.9%. The feeding of the 2% unwashed material is shown in Table 5. C. thermocellum (10% volume) was inoculated at time 0, and T. saccharolyticum (10% volume) was inoculated at time 17 h. MTC was used as fermentation medium. About 17 g/L ethanol was produced in 283 h without added external enzyme, which corresponds to around 50% ethanol yield based on the carbohydrate content in the substrate as shown in FIG. 19.

TABLE 5
Feeding of unwashed mixed hardwoods

| | Time, | Conc., |
|---|---|---|
| Feed | hr | % |
| Starting | 0 | 5 |
| 1 | 85 | 7.5 |
| 2 | 108 | 10 |
| 3 | 162 | 12.2 |
| 4 | 283 | 14.9 |

### Example 13

### Resistance to Contamination by a Co-Culture of C. Thermocellum and T. Saccharolyticum

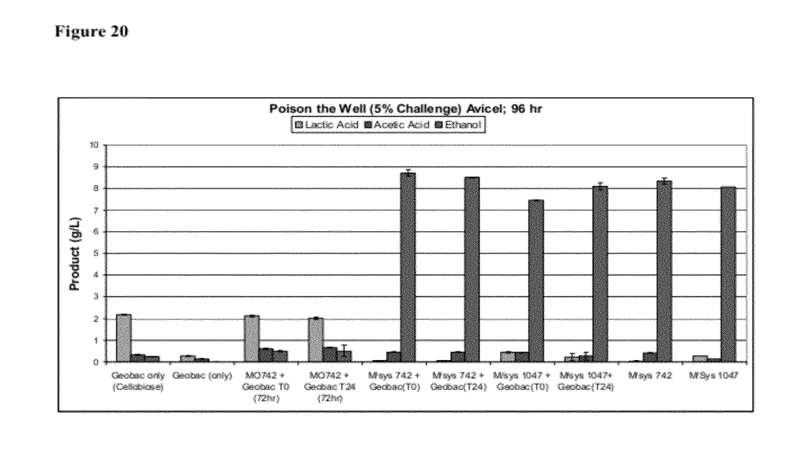

To test the ease of contamination of a co-culture, a known lactic acid bacterium, Geobacillus thermoglucosidasius BAA 1067 (M0057) was introduced at T0 & T24 at 5% of a co-culture between T. saccharolyticum (M01151) & two organic acid KO C. thermocellum strains (lactic and acetate KO) (FIG. 20). The co-culture was spiked at an initial inoculum ration of 95:5, totaling 5% inoculum. (A lower inoculum for Geobacillus was chosen to represent a potentially industrially relevant contaminant floating around a facility). Geobacillus was not capable of hydrolyzing Avicel (20 g/L) alone, but produced lactic acid as the dominant product on cellobiose. When introduced with C. thermocellum Δldh alone at T0 & T24 (5% relative inoculum to C. thermocellum), lactic acid was detected at levels comparable to growth on cellobiose, implying that C. thermocellum alone could readily be contaminated by this organism. Lactic acid formation was a seemingly effective biochemical tracer as it could only be formed by Geobacillus when introduced to co-cultures comprising of the Δldh strain (only detectable levels of acetic acid and ethanol could potentially be produced). Two single organic acid (Δldh & Δpta) knockouts were grown with T. saccharolyticum strain followed by Geobacillus being introduced into the co-cultures at time 0 h and 24 h at a similar 5% ratio. Little to no lactic acid was ever detected in the co-cultures, and the product ratios were quite similar to cultures where Geobacillus was never introduced. Perhaps due to rapid and effective sugar utilization, or other reasons not currently understood, Geobacillus could not gain a foothold in the system after 96 hours. The theoretical ethanol yield in the co-cultures was in the range 65-75% and overall total product yield was in the range 70-80%.

### Example 14

### Continuous Transfer of Co-Culture of Engineered C. Thermocellum and T. Thermosaccharolyticum on 1% Unwashed Pretreated Hardwood

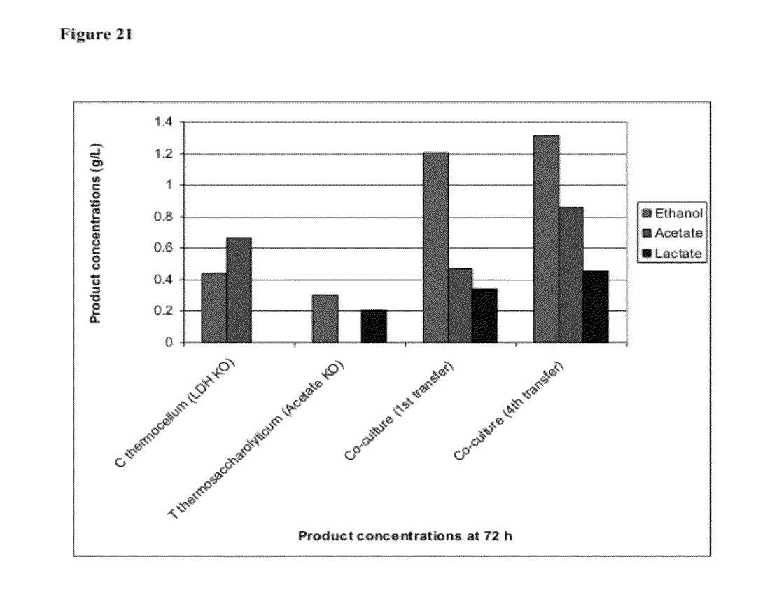

A co-culture of C. thermocellum and T. thermosaccharolyticum was performed on 1% unwashed MS149. Fermentations were performed using the media composition in Table 4 (CTFUD medium composition) and the initial pH of the fermentation was 7. The total product concentration after 72 h of fermentation is shown in FIG. 21. From FIG. 21, the co-cultures produced more ethanol compared to each of the individual mono-cultures. The total product yield was 26% for the monoculture of C. thermocellum and 11% for T. thermosaccharolyticum. For the co-cultures the total yield was 48% on the first transfer and 62% on the 4^{th} transfer. 5% (vol:vol) inoculum was used for each transfer. Both organisms were capable of producing ethanol, but in this consortium, only C. thermocellum could produce acetic acid whereas T. thermosaccharolyticum could produce lactic acid. As seen in FIG. 21, the two organisms appear to be stable after the fourth transfer based on organic acid production and product yield with potentially improved yield after four transfers.

### Example 15

### Continuous Transfer of a Co-Culture on Avicel, Xylan, and Xylose

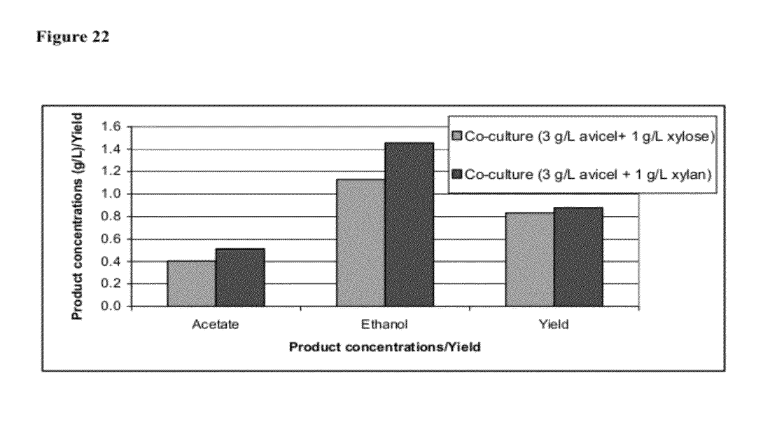

A co-culture of C. thermocellum LDH KO and T. saccharolyticum was maintained by serial passage on MTC (-yeast extract, pH 6.3) with 3 g/L Avicel and 1 g/L Beechwood xylan for a period of 3 months at pH 6.3-6.8. The medium used for the transfers was MTC containing 10 g/L MOPS. Transfers were generally made every 48 hours, although occasional 72 hour transfers also remained viable. During the three month period, the co-culture remained stable as evidenced by visible conversion of Avicel and periodic growth tests on MTC with cellobiose or xylose. A similarly stable line was maintained on 3 g/L Avicel, 1 g/L xylose. The product concentrations and yield form the transfers are shown in FIG. 22. From FIG. 22, the co-cultures Avicel+xylan generated more products compared to the co-culture of Avicel+xylose. The 48 h yield was higher than 80% in both cases.

### Example 16

### Exopolysaccharides May be Responsible for Improved Yield in a Co-Culture

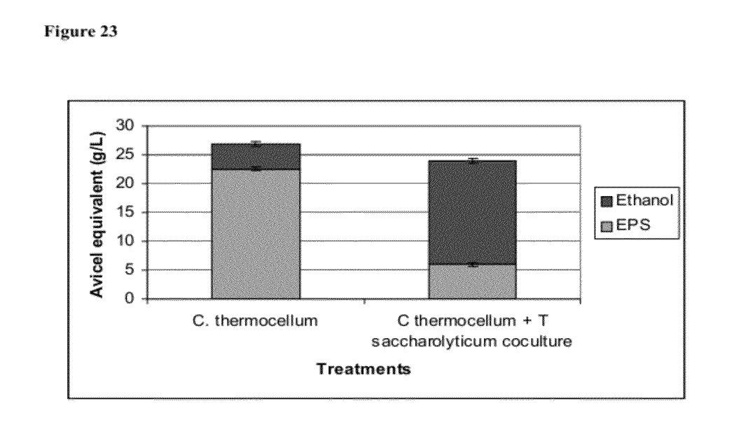

This experiment was performed to determine if the improvements in the yield from co-culture were due to production of exopolysaccharides (EPS). Fermentations were performed in serum bottles using 20 g/L Avicel and 6.5 g/L of birchwood xylan. Fermentations were started at a temperature of 60° C. and an initial pH of 7.5 in serum bottles using the medium in Table 4. FIG. 23 shows the contribution of ethanol and EPS to the total substrate used in the fermentation. The data show that a large portion of the initial substrate was used to produce EPS whereas a significant portion of the substrate went into ethanol production in the case of the co-culture.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for converting lignocellulosic biomass to ethanol, comprising the step of contacting the lignocellulosic biomass with a mixture for a period of time at an initial temperature and an initial pH, thereby producing an amount of ethanol; wherein the mixture comprises a first microorganism and a second microorganism; the first microorganism is a thermophilic or mesophilic microorganism; and the second microorganism is a thermophilic or mesophilic microorganism.

2. The method of claim 1, wherein the second microorganism comprises at least one genetic modification.

3. The method of claim 2, wherein the second microorganism comprises a native gene or a particular polynucleotide sequence that has been partially, substantially, or completely deleted, silenced, inactivated, or down-regulated, which gene or polynucleotide sequence encodes for an enzyme that confers upon the microorganism the ability to produce organic acids as fermentation products; thereby increasing the ability of the second microorganism to produce ethanol as a fermentation product.

4. The method of claim 2, wherein the second microorganism comprises (a) a first native gene that has been partially, substantially, or completely deleted, silenced, inactivated, or down-regulated, which first native gene encodes a first native enzyme involved in the metabolic production of an organic acid or a salt thereof; and (b) a first non-native gene that has been inserted, which first non-native gene encodes a first non-native enzyme involved in the metabolic production of ethanol; thereby increasing the ability of the second microorganism to produce ethanol as a fermentation product.

5. The method of claim 2, wherein the second microorganism comprises (a) a first native gene that has been partially, substantially, or completely deleted, silenced, inactivated, or down-regulated, which first native gene encodes a first native enzyme involved in the metabolic production of an organic acid or a salt thereof; and (b) a first non-native gene that has been inserted, which first non-native gene encodes a first non-native enzyme involved in the hydrolysis of a polysaccharide; thereby increasing the ability of said second microorganism to produce ethanol as a fermentation product.

6. The method of claim 1, wherein the first microorganism is a cellulolytic microorganism.

7. The method of claim 1, wherein the second microorganism is a xylanolytic microorganism.

8-11. (canceled)

12. The method of claim 1, wherein the first microorganism is native Clostridium thermocellum.

13. The method of claim 1, wherein the second microorganism is a genetically engineered Thermoanaerobacterium saccharolyticum.

14. (canceled)

15. The method of claim 1, wherein the first microorganism is a xylanolytic microorganism.

16. The method of claim 1, wherein the second microorganism is a cellulolytic microorganism.

17-20. (canceled)

21. The method of claim 1, wherein the first microorganism is native Thermoanaerobacterium saccharolyticum.

22. The method of claim 1, wherein the second microorganism is a genetically engineered Clostridium thermocellum.

23. (canceled)

24. The method of claim 1, wherein the amount of ethanol produced is at least about 60% of the theoretical yield based on the amount of lignocellulosic biomass metabolized.

25-27. (canceled)

28. The method of claim 1, wherein the period of time is about 10 hours to about 300 hours.

29-32. (canceled)

33. The method of claim 1, wherein the initial temperature is about 30° C. to about 75° C.

34-36. (canceled)

37. The method of claim 1, wherein the initial pH is between about 5 and about 9.

38-69. (canceled)

70. The method of claim 1, wherein the first microorganism is Clostridium thermocellum; the second microorganism is Thermoanaerobacterium saccharolyticum; the initial temperature is about 60° C.; and the initial pH is about 7 or 7.5.

71. (canceled)

72. The method of claim 1, wherein the first microorganism is Clostridium thermocellum; the second microorganism is Thermoanaerobacterium saccharolyticum; the ack gene of the Thermoanaerobacterium saccharolyticum has been partially, substantially, or completely deleted, silenced, inactivated, or down-regulated, thereby producing a genetically-modified Thermoanaerobacterium saccharolyticum; the initial temperature is about 60° C.; and the initial pH is about 7.

73. The method of claim 1, wherein the first microorganism is Thermoanaerobacterium saccharolyticum; the second microorganism is Clostridium thermocellum; the ldh gene of the Clostridium thermocellum has been partially, substantially, or completely deleted, silenced, inactivated, or down-regulated, thereby producing a genetically-modified Clostridium thermocellum; the initial temperature is about 60° C.; and the initial pH is about 7.5.

74. (canceled)

75. The method of claim 1, wherein the lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, and softwood.

76. (canceled)

77. The method of claim 1, wherein said lignocellulosic biomass is selected from the group consisting of corn stover, sugarcane bagasse, switchgrass, and poplar wood.

78-86. (canceled)
